SEQUENCE LISTING (10 sequences)

SEQ ID NO: 1 - protein, 6 aa, synthetic construct
  REGION 1..6: note = complementarity determining region (CDR)
QNVDTN

SEQ ID NO: 2 - skipped

SEQ ID NO: 3 - protein, 9 aa, synthetic construct
  REGION 1..9: note = complementarity determining region (CDR)
QQYNNYPYT

SEQ ID NO: 4 - protein, 8 aa, synthetic construct
  REGION 1..8: note = complementarity determining region (CDR)
GFTFSTYG

SEQ ID NO: 5 - protein, 8 aa, synthetic construct
  REGION 1..8: note = complementarity determining region (CDR)
INGNGGSA

SEQ ID NO: 6 - protein, 15 aa, synthetic construct
  REGION 1..15: note = complementarity determining region (CDR)
VRERDYYGNSRYFDV

SEQ ID NO: 7 - DNA, 322 bp, synthetic construct
  misc_feature 1..322: note = exemplary nucleic acid sequence of the light chain of antibody directed to AXL
  source 1..322: mol_type = other DNA
gacattgtgatgacccagtctcaaaaattcatgtccacatcagtgggagacagggtcagcgtcacctgcaaggccagtcagaatgtggatactaatgtggcctggtatcaacagaaaccaggacaatctcctaaaccactgattttctcggcattcctccggtacagtggagtccctgatcgcttcacaggcagtggatctgggacagatttcagtctcaccgtcagcaatgtgcagtctgaagacttggcagagtatttctgtcagcaatataacaattatccgtacacgttcggaggggggaccaagctggaaatgaaac

SEQ ID NO: 8 - protein, 107 aa, synthetic construct
  REGION 1..107: note = exemplary amino acid sequence of the light chain of antibody directed to AXL
DIVMTQSQKFMSTSVGDRVSVTCKASQNVDTNVAWYQQKPGQSPKPLIFSAFLRYSGVPDRFTGSGSGTDFSLTVSNVQSEDLAEYFCQQYNNYPYTFGGGTKLEMK

SEQ ID NO: 9 - DNA, 366 bp, synthetic construct
  misc_feature 1..366: note = exemplary nucleic acid sequence of the heavy chain of antibody directed to AXL
  source 1..366: mol_type = other DNA
gaggaacagttggtggagtctgggggaggcttagtgcagcctggagggtccctgaaactctcctgtgcagcctctggattcactttcagtacctatggcatgtcttgggttcgccagactccagacaagaggctggagttggtcgcaaccataaatggtaatggtggtagtgcctattatccagacagtgtgaagggccgattcaccatatccagagacaacggcaagaacaccctgtacctgcaaatgagcagtctgaagtctgaggacacagccatgtattactgtgtaagagagagggattattacggtaatagtcgctacttcgatgtctggggcgcagggaccgcggtcaccgtctcctca

SEQ ID NO: 10 - protein, 120 aa, synthetic construct
  REGION 1..120: note = exemplary amino acid sequence of the heavy chain of antibody directed to AXL
EEQLVESGGGLVQPGGSLKLSCAASGFTFSTYGMSWVRQTPDKRLELVATINGNGGSAYYPDSVKGRFTISRDNGKNTLYLQMSSLKSEDTAMYYCVRERDYYGNSRYFDVWGAGTAVTV

(19) United States
(12) Patent Application Publication
(10) Pub. No.: US 2024/0156959 A1
(43) Pub. Date: May 16, 2024

(54) COMBINATION THERAPY FOR THE TREATMENT OF CANCER COMPRISING AN ANTl-EGFR ANTIBODY AND AN AXL-INHIBITOR

(71) Applicant: Yeda Research and Development Co. Ltd., Rehovot (IL)

(72) Inventors: Yosef YARDEN, Rehovot (IL); Ashish NORONHA, Rehovot (IL)

(73) Assignee: Yeda Research and Development Co. Ltd., Rehovot (IL)

(21) Appl. No.: 18/544,514

(22) Filed: Dec. 19, 2023

Related U.S. Application Data
(63) Continuation of application No. PCT/IL2022/050660, filed on Jun. 20, 2022.
(60) Provisional application No. 63/214,371, filed on Jun. 24, 2021.

(51) Int. Cl. A61K 39/395; A61K 31/502; A61K 31/506; A61P 35/00

(52) U.S. Cl. CPC A61K 39/3955; A61K 31/502; A61K 31/506; A61P 35/00

(57) ABSTRACT

A method of treating cancer in a subject in need thereof is disclosed wherein cancer cells of the subject express an EGFR having a mutation in a kinase domain of the receptor. The method comprises administering to the subject a therapeutically effective amount of: (i) an anti-epidermal growth factor receptor (anti-EGFR) antibody; (ii) an AXL inhibitor, and optionally (iii) a tyrosine kinase inhibitor (TKI). Also disclosed is a new anti-AXL antibody.

### RELATED APPLICATIONS

This application is a Continuation of PCT Patent Application No. PCT/IL2022/050660 having International filing date of Jun. 20, 2022, which claims the benefit of priority under 35 USC § 119(e) of U.S. Provisional Patent Application No. 63/214,371 filed on Jun. 24, 2021. The contents of the above applications are all incorporated by reference as if fully set forth herein in their entirety.

### SEQUENCE LISTING STATEMENT

The XML file, entitled 98361SequenceListing.xml, created on Nov. 7, 2023, comprising 13,187 bytes, submitted concurrently with the filing of this application is incorporated herein by reference.

### FIELD AND BACKGROUND OF THE INVENTION

The present invention, in some embodiments thereof, relates to combination treatments for treating cancer and more specifically to those resistant to a tyrosine kinase inhibitor.

The ErbB family of receptor tyrosine kinases, which includes epidermal growth factor receptor (EGFR, also termed ErbB-1, HER1), HER2 (ErbB-2), HER3 (ErbB-3) and HER4 (ErbB-4) is widely known and researched. The ErbB family members and their multiple ligand molecules form a layered signaling network, which is implicated in several human cancers. ErbB activation leads to downstream stimulation of several signaling cascades, including MAPK and PI(3)K/Akt that influence cell proliferation, angiogenesis, invasion and metastasis [Citri and Yarden Nat Rev Mol Cell Biol. (2006) 7(7):505-16]. Because of their oncogenic potential and accessibility, ErbB proteins have emerged as attractive targets for pharmaceutical interventions. Consistently, strategies able to interfere with ErbB functions, such as monoclonal antibodies (mAbs) and tyrosine kinase inhibitors (TKIs), have yielded in the last decade several oncology drugs which have shown great success in treating many patients with lung, breast, colon and other types of cancer.

For example, the anti-EGFR mAbs cetuximab (Erbitux®) and the anti-HER2 mAb trastuzumab (Herceptin®) have been developed and approved for the treatment of human cancers.

TKIs are small-molecule therapeutics designed to bind to the ATP-binding site of the tyrosine kinase domain, preempting the binding of ATP and directly inhibiting the kinase activity of ErbB receptors such as EGFR or HER2. For example, a number of TKIs for EGFR have been developed; including gefitinib (Iressa) and erlotinib (Tarceva®) have recently gained FDA approval in oncology treatment. In addition, TKIs that simultaneously target multiple ErbB species, such as CI-1033 (PD183805) and lapatinib (GW572016/Tykerb®), have also been developed.

However, while many cancer patients were found sensitive to ErbB-targeted therapy, many other patients are resistant to treatment, and even among the initially responsive patients a large percentage experience tumor recurrence and become refractory to therapy. Thus for example, despite initial dramatic response of non-small cell lung cancer (NSCLC) patients to TKIs, all patients acquire resistance within approximately one year. The most common (>50%) mechanism of this acquired resistance involves a specific second site mutation in the EGFR kinase domain. A threonine-to-methionine substitution at position 790 creates a steric hindrance that limits the binding of the TKIs, while preserving the kinase activity. [Wang and Greene J Clin Invest. (2008) 118(7): 2389-2392]. Amplification of the gene encoding another receptor tyrosine kinase, MET, occurs in 5-10% of cases of acquired resistance.

To overcome TKI resistance, several second and third generation TKIs are being developed. Alternatively, a clinical trial combining cetuximab and chemotherapy (cis-platin/vinorelbin) demonstrated a relatively small, but significant increase in patient survival.

Background art includes US Patent Application No. 2019/0231778; US Patent Application No. 2010/0255004; US Patent Application No. 2019/022420; US Patent Application No. 2018/0153888; International Patent Application No. 2011/014872, International Patent Application No. 2018/183944, U.S. Pat. Nos. 7,939,072; 10,028,956 and 10,028,958.

### SUMMARY OF THE INVENTION

According to an aspect of the present invention there is provided a method of treating cancer in a subject in need thereof comprising administering to the subject a therapeutically effective amount of:
(i) an anti-epidermal growth factor receptor (anti-EGFR) antibody;
(ii) a tyrosine kinase inhibitor (TKI); and
(iii) an AXL inhibitor,
wherein cancer cells of the subject express an EGFR having a mutation in a kinase domain of the receptor, thereby treating the cancer in the subject.

According to an aspect of the present invention there is provided a pharmaceutical composition comprising an antibody that specifically binds to AXL as an active agent and a pharmaceutically acceptable carrier, the antibody comprising an antigen recognition domain having complementarity determining region (CDR) amino acid sequences as set forth in: SEQ ID NOs: 1 (CDR1), SAF (CDR2) and 2 (CDR3), sequentially arranged from N to C on a light chain of the antibody) and SEQ ID NOs: 4 (CDR1), 5 (CDR2) and 6 (CDR3), sequentially arranged from N to C on a heavy chain of the antibody.

According to an aspect of the present invention there is provided an article of manufacture comprising:
(i) an anti-epidermal growth factor receptor (anti-EGFR) antibody;
(ii) a tyrosine kinase inhibitor (TKI); and
(iii) an AXL inhibitor.

According to an embodiment of the present invention, the mutation comprises a substitution of Threonine to Methionine at position 790 (T790M).

According to an embodiment of the present invention, the cancer is resistant to the TKI when provided as a monotherapy.

According to an embodiment of the present invention, the treating is a first line treatment.

According to an embodiment of the present invention, the treating is a second line treatment.

According to an embodiment of the present invention, the TKI is provided below gold standard dosing as a single agent.

According to an embodiment of the present invention, the cancer is lung cancer.

According to an embodiment of the present invention, the lung cancer is non-small cell lung cancer (NSCLC).

According to an embodiment of the present invention, the subject is:
(i) a non-smoker;
(ii) a female; and/or
(iii) of Asian ethnicity.

According to an embodiment of the present invention, the anti-EGFR antibody is cetuximab.

According to an embodiment of the present invention, the TKI is osimertinib, perlitinib (EKB-569), neratinib (HKI-272), canertinib (CI-1033), vandetanib (ZD6474), afatinib, dacomitinib, rociletinib (CO-1686), HM61713, Gefitinib, Erlotinib, Icotinib and WZ4002.

According to an embodiment of the present invention, the TKI is osimertinib.

According to an embodiment of the present invention, the AXL inhibitor is an antibody which specifically binds to AXL.

According to an embodiment of the present invention, the AXL inhibitor is a AXL-specific tyrosine kinase inhibitor (e.g. small molecule inhibitor).

According to an embodiment of the present invention, the antibody comprises an antigen recognition domain having complementarity determining region (CDR) amino acid sequences as set forth in: SEQ ID NOs: 1 (CDR1), SAF (CDR2) and 2 (CDR3), sequentially arranged from N to C on a light chain of the antibody and SEQ ID NOs: 4 (CDR1), 5 (CDR2) and 6 (CDR3), sequentially arranged from N to C on a heavy chain of the antibody.

According to an embodiment of the present invention, the method further comprises confirming that the cancer cells of the subject express the mutation prior to the administering.

According to an embodiment of the present invention, the pharmaceutical composition further comprises an anti-epidermal growth factor receptor (anti-EGFR) antibody and/or a tyrosine kinase inhibitor (TKI).

According to an embodiment of the present invention, the AXL inhibitor is an antibody comprising an antigen recognition domain having complementarity determining region (CDR) amino acid sequences as set forth in: SEQ ID NOs: 1 (CDR1), SAF (CDR2) and 2 (CDR3), sequentially arranged from N to C on a light chain of the antibody) and SEQ ID NOs: 4 (CDR1), 5 (CDR2) and 6 (CDR3), sequentially arranged from N to C on a heavy chain of the antibody.

According to an embodiment of the present invention, the article of manufacture is for use in treating cancer of a subject, wherein the subject has cancer cells which express an EGFR having a mutation in a kinase domain of the receptor.

According to an embodiment of the present invention, the cancer is lung cancer.

According to an embodiment of the present invention, the lung cancer is non-small cell lung cancer (NSCLC).

According to an embodiment of the present invention, the subject is a non-smoker.

According to an embodiment of the present invention, the anti-EGFR antibody is cetuximab.

According to an embodiment of the present invention, the According to an embodiment of the present invention, the TKI is osimertinib, perlitinib (EKB-569), neratinib (HKI-272), canertinib (CI-1033), vandetanib (ZD6474), afatinib, dacomitinib, AZD9291, rociletinib (CO-1686), HM61713 and WZ4002.

According to an embodiment of the present invention, the TKI is osimertinib.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G: Upregulation of AXL is essential for resistance and tolerance. (A) PC9 cells (2×10⁶) were subcutaneously implanted in nude mice. When tumors became palpable, mice were randomized into groups (n=8) that were daily treated with erlotinib (10 mg/kg/day) or osimertinib (5 mg/kg/day). Tumor volumes are presented (average ±SEM). (B) 3-4 mice from each group presented in A were euthanized when tumors reached 1,500 mm 3 Tumors were extracted and subjected to immunoblotting. Tubulin (TUB) served as the loading control. (C and D) AXL-overexpressing (C) and AXL-knockout PC9 cells (2×10⁶, D), along with naïve cells, were subcutaneously implanted in mice. When tumors became palpable, mice were randomized and treated as in A. (E) Data were re-analyzed for AXL mRNA levels in 23 patients with EGFR+NSCLC. Specimens (total=53) were collected at several time points, prior to and after treatment with osimertinib. (F and G) AXL-overexpressing (OX; F) or AXL-knockout PC9 cells (KO; G), along with naïve cells, were untreated or treated with TKIs (1 or 3 μM). Drugs and media were refreshed on day 3 and 6. On day 9, remaining cells (DTPs) were fixed, stained and quantified. Averages±S.D. (triplicates) are shown.

FIGS. 2A-G: TKIs induce DNA breaks and AXL restrains this effect. Naïve and AXL-overexpressing (OX) PC9 cells were used. (A) Cells were incubated for 72 hours with TKIs (10 nM) and viability was determined (means±S.D., three experiments). (B) Cells were treated for 48 hours with TKIs (1 μM), and the fractions undergoing early/late apoptosis were determined (means ±S.D., triplicates). (C) Cells were treated as in B, extracted and probed for the indicated proteins using immunoblotting. (D) Cells were treated as in A, fixed and stained for gamma-H2A.X (green), actin (red) or DAPI (blue). Scale bars, 50 μm. Dot plots represent average foci counts per nucleus. (E) Cells were incubated with TKIs (1 μM) for 24 hours. DNA damage was estimated using the comet assay (upper panels). The lower panels present the scores of 40 comets. Results represent means (±S.D.) from three experiments. (F) Cells were treated for 48 hours as in A, and ROS levels were assayed. Data are means (±S.D., three experiments). (G) Cells stably expressing pDRGFP were transfected with pCBASce-I and then treated as in A, prior to flow cytometry. A mock transfection was used for normalization. Quantification of relative HR capacities is presented (means ±S.D. from three experiments).

FIGS. 3A-G. AXL and RAD18 are functionally linked. (A) Extracts of tumors presented in FIG. 1C were immunoblotted. (B) PC9 cells were treated with erlotinib (1 μM), or DMSO and RNA was subjected to real time PCR. (C) Transcriptomic data corresponding to 506 patients with lung adenocarcinoma were used to generate an expression correlogram of 19 DNA damage response genes. Pearson correlation coefficients are indicated in each square (see scale code). (D) Parental and RAD18 knockout cells were treated for 9 days with a TKI. Fractions of surviving DTPs are shown. (E) Naïve and RAD18-knockout cells were probed using anti-AXL or anti-RAD18 antibodies. Thereafter, the cells were processed for proximity ligation analysis (red signals). Representative images and PLA quantification are shown. Bar, 20 μm. (F) Cells were treated for 2-9 days with erlotinib (1 μM) or H₂O₂ (40 μM). Extracts were subjected to immunoprecipitation with an anti-RAD18 or control antibody and immunoprecipitates were immunoblotted. (G) Naïve and AXL-overexpressing cells, along with PC9ER cells, were treated with erlotinib (1 μM). Genomic DNA was isolated after 9 days and the T790M point mutation was determined using digital PCR. Shown are fraction of T790M in cellular DNA. Data are means ±SEM (4 experiments).

FIGS. 4A-K. AXL regulates purine metabolism and mutational bias. (A) RNA from PC9 cells and AXL-KO cells was sequenced and differentially expressed genes were subjected to KEGG Pathway Enrichment analysis. (B) The indicated cell extracts were probed using immunoblotting. (C) A waterfall plot depicting fold changes in metabolite abundance in AXL-KO and PC9 cells. Metabolites were rank ordered and specific compounds are indicated. (D) Fractional labeling of IMP in PC9-KO cells using [amide-15N]glutamine and 24 hours of incubation. (E) Fractional labeling of purines determined in AXL-KO and naive cells incubated for 24 hours with [U-13C]glucose (means ±S.D., 3 experiments). (F) An AXL expression vector and MYC promoter luciferase reporter plasmid were transfected into HEK293 cells. Renilla was used as control. Luminescence reading was taken 48 hours later. (G) PPAT and PAICS luciferase reporter plasmids were transfected into HEK293 cells, along with AXL and MYC vectors. Luminescence readings (means±S.D., 3 experiments) were normalized to a GAPDH reporter. (H-J) Purine mutational bias was analyzed in the TCGA lung adenocarcinoma dataset (n=506) and presented versus EGFR (H), AXL expression (I) and EGFR or RAS mutations (J). (K) A core MYC gene expression signature was analyzed against level of AXL expression in the cohort of 506 patients.

FIGS. 5A-E. Combining an anti-AXL antibody and EGFR blockers prevents tumor relapses. (A-C) PC9 cells (3×10⁶) were implanted in CD1-nu/nu mice. When tumors became palpable, mice were randomized into groups of 8 animals that were treated (hatched area) with the indicated antibodies (total dose: 0.2 mg/mouse/injection) once every three days, or with osimertinib (5 mg/kg/day). Data are means ±SEM. (A: single agent, B: dual therapy, and C: triple or quadruple drug combinations). (D) The PDX tumor model TM00193 was implanted in NSG mice. When tumors became palpable, mice were randomized into groups (n=7), which were treated (hatched area) with the indicated antibodies (see A) or with osimertinib (10 mg/kg/day). Tumor volumes (averages ±SD) are indicated. (E) PC9 cells (3×10⁶) were subcutaneously implanted in CD1-nu/nu mice. When tumors became palpable, mice were treated with osimertinib (5 mg/kg/day) and after tumors regressed and relapsed (300 mm³) animals were randomized into groups (n=7) that were treated (hatched area) with the indicated drugs as in A. Panels E1-E6 show tumor volumes in individual mice that were initially treated with osimertinib (5 mg/kg/day) and later switched them to the triple drug combination (3×). All treatments were terminated once tumors disappeared. CTX, cetuximab, Osi, osimertinib.

FIGS. 6A-H. AXL activation by TKIs and establishment of AXL-overexpressing and AXL-knockout cells. (A and B) PC9 cells were treated with erlotinib (25 nM), osimertinib (100 nM), or solvent (DMSO) for different time intervals. Thereafter, cell extracts were subjected to immunoblotting. (C) PC9 cells were treated as in A for different time intervals, as indicated. Thereafter, cell extracts were subjected to RPPA using pre-validated antibodies to the indicated proteins. (D) PC9 cells were treated for 48 hours as in A and extracts analyzed using flow cytometry with anti-AXL antibodies. Normalized AXL surface levels are shown (means ±S.D. values from three experiments). (E and G) PC9 cells overexpressing AXL (E) and AXL-knockout cells (G), along with the parental cells, were grown in complete media for 24 hours, lysed and the indicated proteins probed using immunoblotting. Tubulin served as loading control. (F and H) PC9 cells overexpressing AXL (F), AXL-knockout cells (H) and the parental cells, were allowed to grow in complete media for 24 hours. Thereafter, cells were analyzed for surface levels of AXL, as in D.

FIGS. 7A-G. AXL knockdown enhances TKI-induced apoptosis, ROS generation, comets and histone 2A.X phosphorylation. AXL-knockout (KO) PC9 cells, along with the parental cells, were used in all experiments. (A) Cells were incubated for 72 hours with erlotinib (10 nM) or osimertinib (10 nM) and their viability was determined using the MTT assay. Data are means ±S.D. from three experiments. (B) Cells were treated for 48 hours with erlotinib or osimertinib (each at 1 μM), and the fractions of cells undergoing early or late apoptosis were determined (means ±S.D., triplicates). (C) Cells were treated for 48 hours with erlotinib, osimertinib (each at 1 μM) or solvent and later extracted. The indicated proteins were detected using immunoblotting. Tubulin served as loading control. (D) Cells were seeded on coverslips and later treated as in A. Fixed cells were stained for gamma-H2A.X (green), actin (red) and DAPI (blue). Images were captured using a confocal microscope. Scale bars, 50 μm. The dot plot represents the average counts of foci per nucleus. (E) Cells were incubated for 24 hours with erlotinib or osimertinib (each at 1 μM). DNA damage was estimated using the comet assay (upper panels). The lower panels present the scores of 40 comets corresponding to each condition. Results represent means (±S.D.) from three experiments. (F) Cells were treated for 48 hours as in A, and ROS levels were measured using a luminometer. Data are means (±S.D.) from three experiments. (G) Control and AXL-KO PC9 cells stably expressing pDRGFP were transfected with the pCBASce-I plasmid and then treated as in A, prior to flow cytometry. Mock transfection was used for normalization. Quantification of relative HR capacities is presented in bar graphs. Results represent means (±S.D.) from three experiments.

FIGS. 8A-H. RAD18 binds with AXL and undergoes neddylation. (A) PC9 cells were treated with erlotinib (1 μM) or DMSO for 1-9 days. Thereafter, cells were analyzed using immunoblotting. (B) PC9 and H1975 cells were either untreated or treated for 9 days with erlotinib or osimertinib at the indicated concentrations. Control cells were treated with DMSO. Thereafter, the cells were extracted and the indicated proteins detected using immunoblotting. Tubulin served as loading control. (C) RAD18-knockout cells (two clones), along with the PC9 parental cells, were extracted and the indicated proteins detected by means of immunoblotting. (D) PC9 and RAD18-knockout cells were incubated for 72 hours with erlotinib or osimertinib (15 nM). Cell viability was determined using the MTT assay (means ±S.D.; three experiments. (E) AXL-knockout, RAD18-knockout and parental PC9 cells were treated with erlotinib (1 μM) for 2-9 days. Next, cells were extracted and the analyzed using immunoblotting. Tubulin served as loading control. (F) PC9 and derivative AXL-overexpressing cells were extracted and the extracts were subjected to immunoprecipitation with an anti-RAD18 (upper panel) or an anti-AXL (lower panel) antibody. The immunoprecipitates and whole extracts (Input) were immunoblotted as indicated. (G) HEK293 cells were transfected with vectors encoding AXL-GFP, GFP alone and MYC-tagged NEDD8. Cell extracts were prepared 48 hours later and subjected to immunoprecipitation of RAD18, followed by immunoblotting with antibodies specific to either MYC or RAD18. (H) HEK293 cells were transfected with vectors encoding AXL-GFP (increasing amounts), GFP alone, or HA-tagged ubiquitin, as indicated. Cell extracts were prepared 48 hours later and subjected to immunoprecipitation of RAD18, followed by immunoblotting with antibodies specific to either RAD18 or the HA tag. Whole extracts (Input) were immunoblotted with the indicated antibodies.

FIGS. 9A-L. AXL controls purine synthesis and metastasis. (A) RNA was isolated from parental PC9 and AXL-knockout cells and real-time PCR was used to determine the relative expression levels of purine pathway genes and genes implicated in metastasis. Normalized levels (means of three independent experiments) are shown. (B and C) The indicated cell lines were allowed to grow in complete media for 48 hours. Thereafter, we performed targeted metabolic analysis using mass spectrometry. The bar graphs represent relative intensity of the indicated purines and pyrimidines in AXL-knockout cells (B) and AXL overexpressing cells (C), relative to the parental cells. Shown are averages of two independent experiments. (D) Cells were treated for 24 hours with DMSO, an AKT inhibitor (capivasertib; 1 μM) or a MYC inhibitor (10058-F4; 1 μM). The cells were lysed and the indicated proteins detected using immunoblotting. (E) ³H-thymidine incorporation assays were performed with the indicated cells (5×10⁴) pre-seeded in 24-well plates. The incubation was stopped at the indicated time points and radioactivity determined. Shown are means ±SEM (3 experiments). (F) Parental or AXL-KO cells were allowed to attain 80% confluency. Following incubation with BrdU (60 min), cells were fixed and subjected to BrdU-FITC labeling and propidium iodide (PI) detection. Shown are the means of cell cycle distributions obtained using flow cytometry. (G) The indicated cell lines were allowed to attain 70% confluency prior to SA-beta gal staining (performed in triplicates). (H) Parental PC9 cells and AXL-knockout cells (2×10⁶) were subcutaneously implanted in CD1-nu/nu mice and tumor growth was monitored. Data are means ±SEM from 8 mice per group. The experiment was repeated twice. (I) The indicated cells were seeded on transwell chambers and following 16 hours of incubation, cells that reached the lower face of the chambers were fixed and stained. The bar plots represent quantification of the relative areas covered by cells. (J) The endogenous steady state activities of RHOA, RAC1 and CDC42 were determined in whole cell extracts using an ELISA-based kit (G-LISA, Cytoskeleton). Shown are averages ±SEM of quadruplicates. (K and L) PC9 parental and AXL-knockout cells (1×10⁶) tagged with luciferase were injected through the tail vein into NSG mice (6 mice per group). Lungs were excised 3 weeks later and analyzed for metastasis using bioluminescence imaging. Shown are whole lung images corresponding to each mouse. The average radiance (photons/sec/cm²/sr) is shown for each group. Each animal is represented by a single data point.

FIGS. 10A-I. A combination of the anti-AXL mAb654, cetuximab and osimertinib cooperatively inhibits cell survival and DTPs. (A) H1299 cells were plated and treated with the anti-AXL 654 mAb (20 μg/ml). At the indicated time intervals, the cells were lysed and the indicated proteins were detected using immunoblotting. Tubulin served as loading control. (B) PC9 cells (5×10⁴) were plated onto 24-well plates and 16 hours later the medium was replaced with serum-free medium containing anti-AXL 654 mAb, or saline (control), at different doses (as indicated), along with 3 H-thymidine (1 μCi). The incubation was stopped 48 hour later and radioactivity incorporated into DNA was determined. Data are means ±SD of three independent experiments. (C and D) PC9 (panel C) and H1975 cells (panel D) were treated for 72 hours with either osimertinib (25 nM) or the indicated mAbs. Control cells were treated with DMSO. Cell viability was estimated using the MTT assay. Data are means ±S.D. values from three independent experiments. (E) PC9 cells were treated for 48 hours as indicated. The bar graph represents the fractions of post treatment early or late apoptosis, as determined using flow cytometry. (F) PC9 cells were treated as indicated for 48 hours and relative ROS levels were determined. Data are means ±S.D. values from three independent experiments. (G) H1975 cells were treated for 48 hours with the indicated drugs. Thereafter, the cells were lysed and specific proteins were detected using immunoblotting. Tubulin served as loading control. (H and I) PC9 (panel H) and H1975 cells (panel I) were treated for 9 days as indicated. The media and inhibitors were refreshed every 3 days. At the end of the ninth day, the cells were fixed and stained with crystal violet. The bar graph represents quantification of DTPs in treated cells relative to the control group.

FIGS. 11A-H. A combination comprising anti-AXL, anti-EGFR and osimertinib cooperatively prevents relapses in animals. (A-D) All data refer to FIGS. 5A-E, panels B through E, respectively (A: monotherapy, B: dual therapy, C: triple or quadruple drug combinations, and D, the PDX tumor model TM00193). Shown are Kaplan-Meier survival curves of control and treated mice. Animals were monitored for up to 250 days. Mice were euthanized when tumor size reached 1,500 mm 3 Data are means ±SEM from 8 mice per group. (E) Tumor-bearing mice were treated as in FIGS. 5A-E. Three mice from each group were euthanized after one week of treatment. Shown are immunoblots of tumor extracts. The antibodies used were specific to receptors and downstream effectors, purine pathway proteins and DNA damage response components. Tubulin served as loading control. (F and G) H1975 cells (3×10⁶) were subcutaneously implanted in CD1-nu/nu mice. When tumors became palpable, mice were randomized into groups of 10 animals each, which were treated for 30 days (hatched area) with the indicated antibodies (total dose: 0.2 mg/mouse/injection), once every three days, or with osimertinib (5 mg/kg/day). Mice were euthanized when tumor size reached 1,500 mm³. Shown are growth curves (F) and Kaplan-Meier survival curves (G). Data are means ±SEM. (H) Kaplan-Meier survival curves corresponding to the PDX model shown in FIG. 5D.

FIGS. 12A-B. Combining the anti-AXL antibody and EGFR blockers prevents relapses of the H1975 and the PDX TM00193 models by means of controlling purine metabolism and DNA replication. (A) Fragments of the TM00193 PDX model were implanted in NSG mice. When tumors became palpable, the mice were treated with osimertinib, cetuximab, anti-AXL mAb654, or with the indicated drug combinations (see FIG. 5D). One mouse from each group was euthanized following one week of treatment. Tumor extracts were resolved using electrophoresis. Shown are immunoblots of the corresponding tumor extracts. Tubulin (TUB) was used as the loading control. (B) Targeted metabolite analysis that used mass spectrometry was applied on the PDX extracts in parallel to the immunoblot analysis. The bar graphs represent relative intensity of IMP, AMP and GMP. Statistical analyses were performed using one-way ANOVA with the Tukey's multiple comparison test (*, P≤0.05; **, P≤0.01; ***, P≤0.001).

FIG. 13 is a graph illustrating that the combination of an AXL inhibitor and anti-AXL antibody is effective at treating osimertinib-resistant lung cancer.

### DESCRIPTION OF SPECIFIC EMBODIMENTS OF THE INVENTION

The present invention, in some embodiments thereof, relates to combination treatments for treating cancer and more specifically to those resistant to a tyrosine kinase inhibitor.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Targeted treatment of cancer consists primarily of tyrosine kinase inhibitors (TKIs) and monoclonal antibodies (mAbs), but the emergence of resistance limits efficacy of both these agents. Nevertheless, despite wide variation in the mechanisms of resistance, many of them coalesce into a few convergences, including de novo mutagenesis and bypass routes. Although the sequence of events preceding establishment of resistant clones is poorly understood, one commonality, which is shared by antibiotic-treated bacteria, entails a transitory state, called drug tolerant persister (DTP). To determine the mechanisms driving transformation of DTP cells to resisters, the present inventors studied lung cancer models that express mutant forms of the epidermal growth factor receptor (EGFR). Three generations of TKIs have been developed to overcome the deleterious effects of EGFR mutations. The majority of patients initially respond to treatments with erlotinib and other first-generation TKIs, but drug resistance inevitably evolves, due mainly to the T790M, MET amplification or stimulation of the transcription-driven EMT (epithelial-mesenchymal transition) program. Second- and third-generation TKIs (e.g., osimertinib), have also been developed, but their application is similarly limited by acquired resistance.

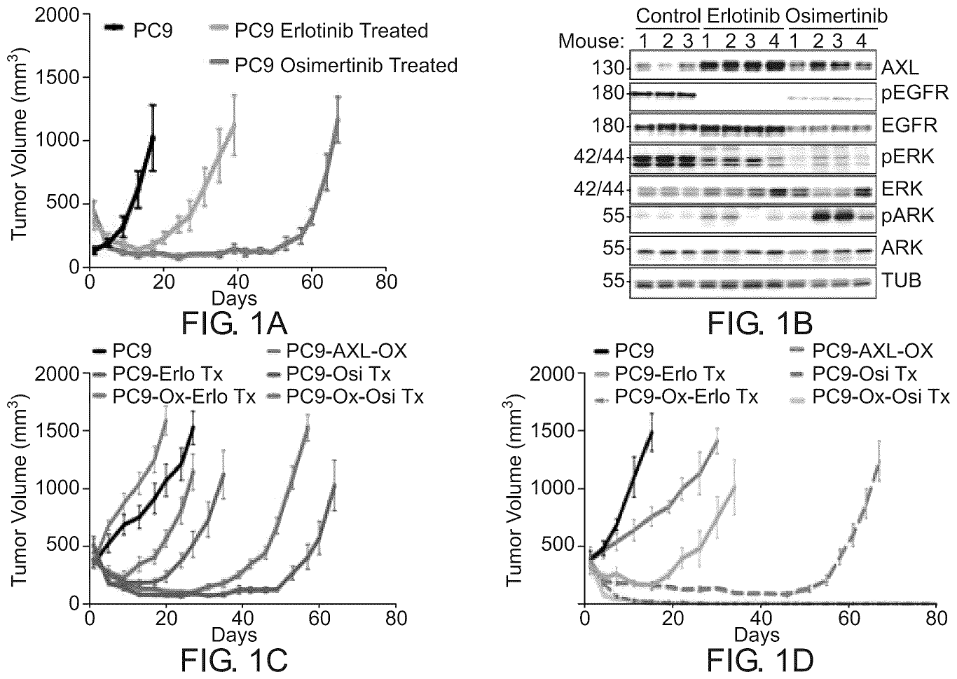

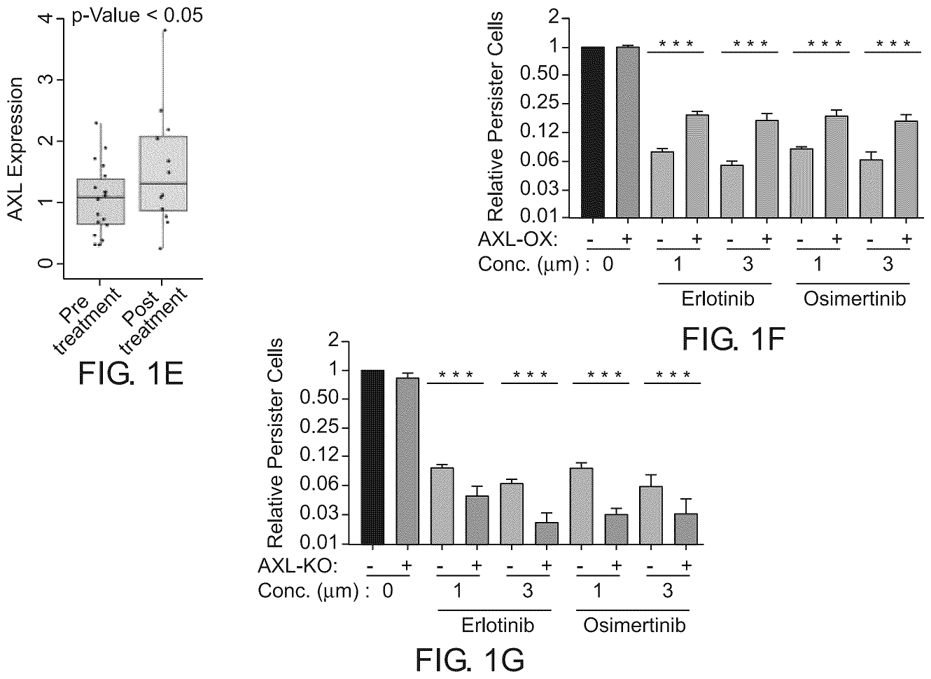

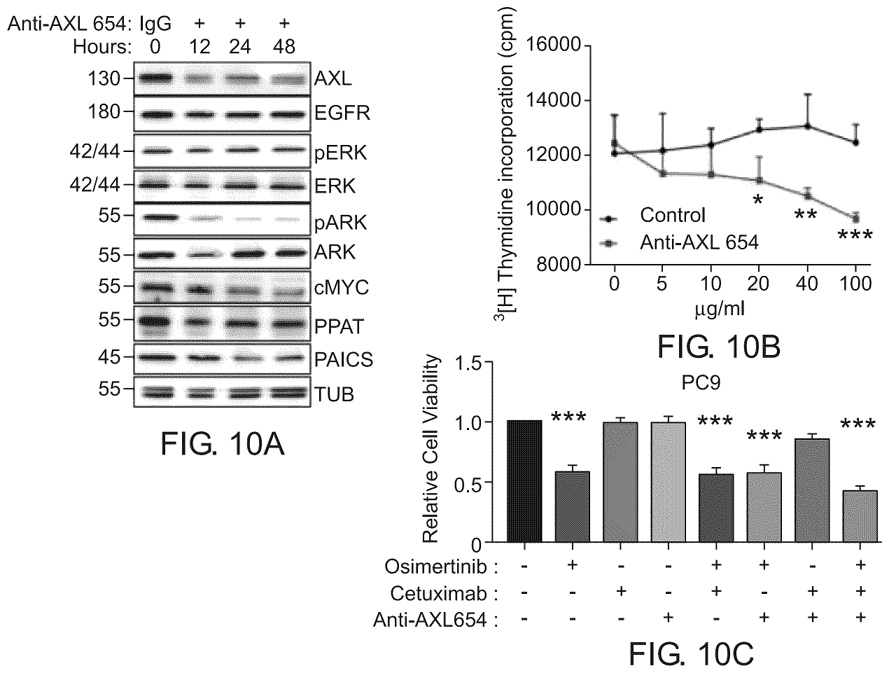

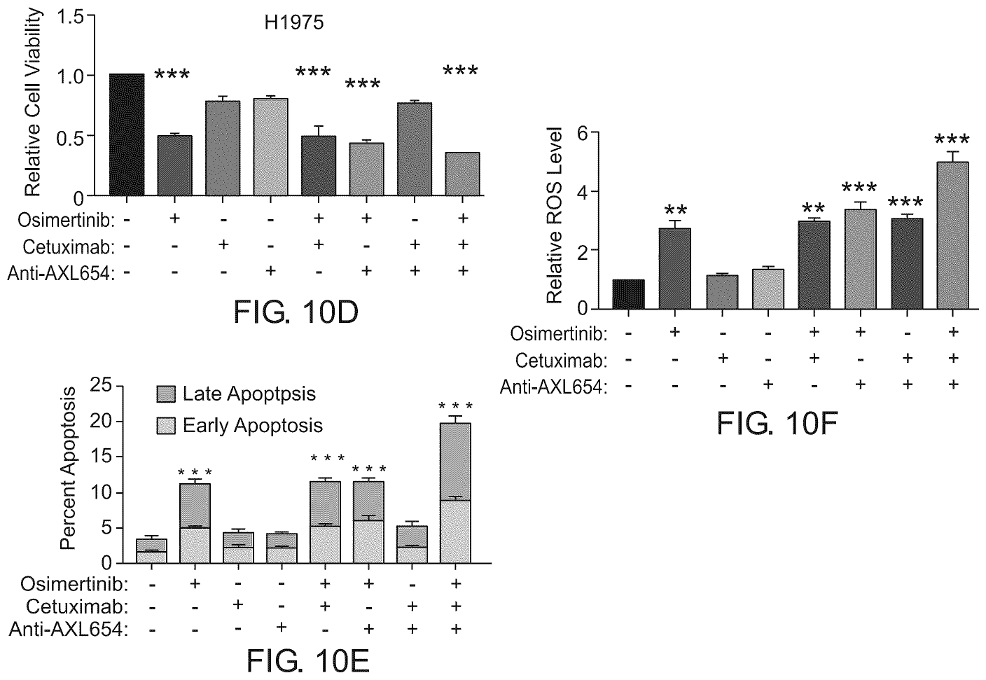

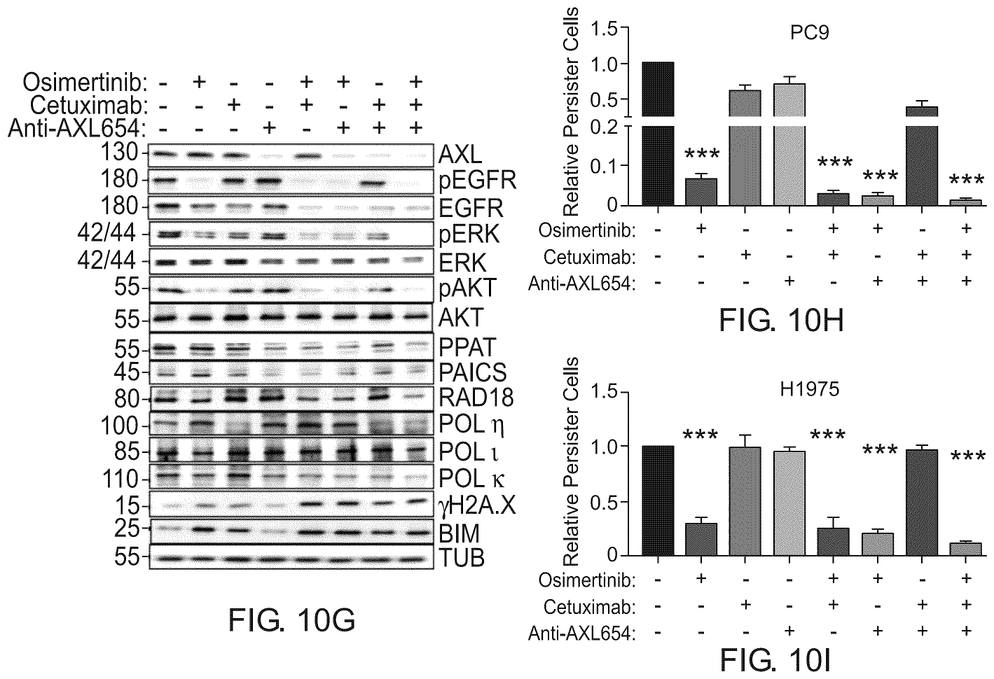

The present inventors have now shown that AXL is activated or up-regulated in response to TKIs and supports survival of drug-treated cells (FIGS. 1A-G). The present inventors generated anti-AXL mAbs and showed that in combination with an anti-EGFR mAb, and TKIs the triplet combination was particularly effective at eliminating DTPs in PC9 and H1975 cells (FIGS. 10A-I).

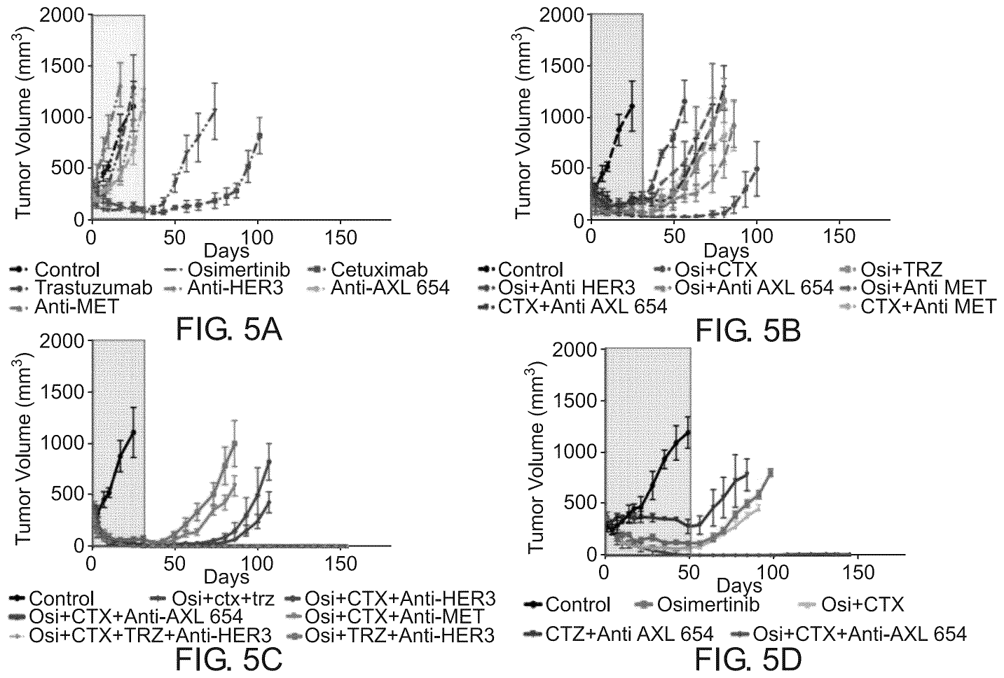

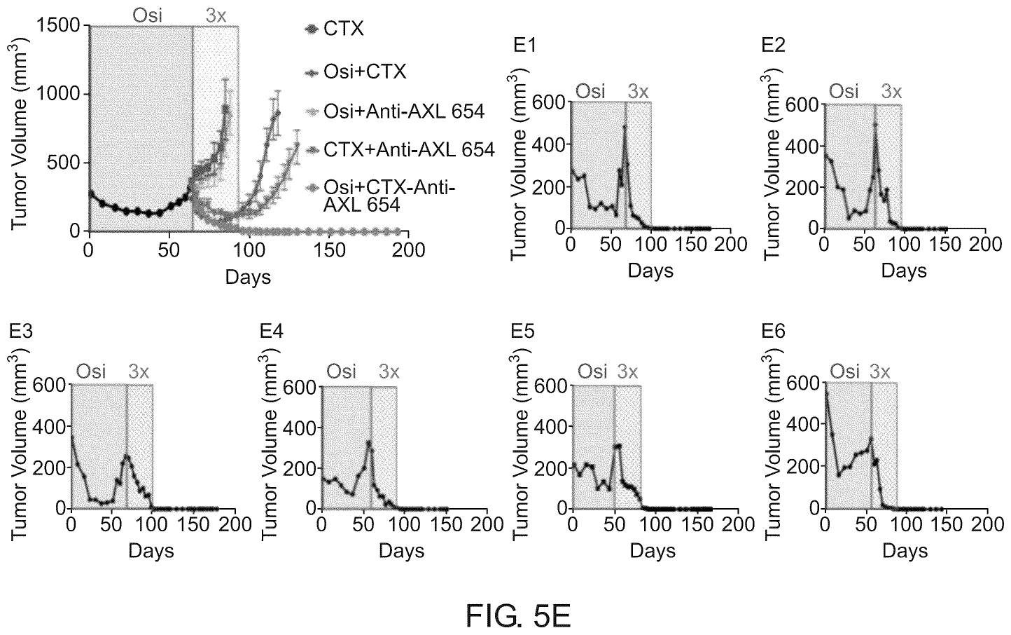

Whilst further reducing the present invention to practice, the present inventors demonstrated that the triplet combination was capable of inhibiting tumor relapses in an animal cancer model, in which PC9 cells were implanted in athymic mice (FIGS. 5A-C and 11A-C).

Accordingly, the present inventors propose that combined use of TKIs, anti-EGFR mAb and anti-AXL inhibitors is an effective way of treating cancer in general and more specifically to cancers that are resistant to TKIs.

Thus, according to an aspect of the present invention there is provided a method of treating cancer in a subject in need thereof comprising administering to the subject a therapeutically effective amount of:
(i) an anti-epidermal growth factor receptor (anti-EGFR) antibody;
(ii) a tyrosine kinase inhibitor (TKI); and
(iii) an AXL inhibitor,
wherein cancer cells of said subject express an EGFR having a mutation in a kinase domain of said receptor, thereby treating the cancer in the subject.

According to another aspect of the present invention, there is provided a method of treating lung cancer in a subject in need thereof comprising administering to the subject a therapeutically effective amount of:
(i) an anti-epidermal growth factor receptor (anti-EGFR) antibody; and
(ii) an AXL inhibitor,
wherein cancer cells of said subject express an EGFR having a mutation in a kinase domain of said receptor, wherein said cancer is resistant to a TKI when provided as a monotherapy, thereby treating the lung cancer in the subject.

As used herein the term “subject” refers to a mammal, preferably a human being at any age which suffers from the pathology.

In one embodiment, the subject is at least one of the following:
(i) a non-smoker;
(ii) a female; and/or
(iii) of Asian ethnicity.

The term “treating” refers to inhibiting or arresting the development of a pathology (disease, disorder or condition, e.g. cancer) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

Tyrosine kinase inhibitors:

As used herein the term “tyrosine kinase inhibitors (TKIs)” refers to a small molecule capable of inhibiting an ErbB signaling pathway. Typically, TKIs as contemplated herein may be categorized to four groups: (1) ATP-competitive inhibitors, which bind predominantly to the ATP-binding site of the kinase when this site is in the active conformation; (2) inhibitors that recognize and bind to the non-active conformation of the ATP-binding site of the kinase, thus making activation energetically unfavorable; (3) allosteric inhibitors, that bind outside of the ATP-binding site, modifying the tridimensional structure of the receptor and disrupting the interaction between the ATP and the kinase pocket; and (4) covalent inhibitors, that bind irreversibly by covalently bonding to the ATP-binding site of the target kinase. The TKI can be specific to a specific ErbB family member or can inhibit multiple ErbB family members. The TKI can recognize wild type ErbB family member and/or a mutated ErbB family member.

Non limiting examples of TKI include osimertinib, erlotinib HCL (OSI-774; Tarceva®; OSI Pharma), gefitinib (Iressa®, AstraZeneca and Teva), lapatinib (Tykerb®, GlaxoSmithKline), canertinib (CI-1033, PD183805; Pfizer), PKI-166 (Novartis); PD158780; pelitinib; AG 1478 (4-(3-Chloroanillino)-6,7-dimethoxyquinazoline), canertinib (CI-1033, PD 183805; Pfizer) and Zactima (ZD6474), perlitinib (EKB-569), neratinib (HKI-272), vandetanib (ZD6474), afatinib, dacomitinib, AZD9291, rociletinib (CO-1686), HM61713 and WZ4002.

According to a specific embodiment, the TKI is a pan-ErbB inhibitor, i.e., capable of binding and inhibiting the kinase activity of more than one ErbB family member, such as lapatinib.

According to specific embodiments the TKI is specific to a single ErbB family member. i.e., does not significantly affect other members in the ErbB family such as an EGFR-specific TKI.

According to specific embodiments the TKI is selected from the group consisting of osimertinib, rociletinib (CO-1686), olmutinib (HM61713), nazartinib (EGF816), naquotinib (ASP8273), mavelertinib (PF-0647775), and AC0010, afatinib, erlotinib, gefitinib and lapatinib.

According to a specific embodiment the TKI is osimertinib.

According to specific embodiments, the TKI is an irreversible TKI. Non-limiting examples of irreversible TKIs include perlitinib (EKB-569), neratinib (HKI-272), canertinib (CI-1033), vandetanib (ZD6474), afatinib and dacomitinib.

According to specific embodiments, the irreversible TKI is typically used when the cancer exhibits resistance to a reversible (or an irreversible) first generation TKI such as osimertinib, erlotinib, gefitinib and lapatinib.

The TKI may be administered at a gold standard dosing as a single agent, below a gold standard dosing as a single agent or above a gold standard dosing as a single agent.

According to specific embodiments, the TKI is administered below gold standard dosing as a single agent.

As used herein the term “gold standard dosing” refers to the dosing which is recommended by a regulatory agency (e.g., FDA), for a given tumor at a given stage.

According to other specific embodiments the TKI is administered at a dose that does not exert at least one side effect which is associated with the gold standard dosing. Non-limiting examples of side effects of a TKI treatment include skin rash, diarrhea, mouth sores, paronychia, fatigue, hyperglycemia, hepatotoxicity, kidney failure, cardiovascular effects, electrolytes anomalies and GI perforations.

Anti-EGFR antibodies:

As used herein “EGF-R” refers to a receptor tyrosine kinase (RTK) of the epidermal growth factor receptor family, also referred to as HER1 and ErbB-1. According to a specific embodiment the EGFR is human EGFR i.e., EGFR HUMAN, P00533.

As used herein the term “cetuximab”, trademarked as Erbitux®, refers to an immunotherapy drug that contains the active ingredient cetuximab, an anti-EGF-R monoclonal antibody.

Additional antibodies known to target EGF-R include Matuzumab, Patitumumab and Necitumumab.

The term “antibody” as used herein includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

According to a specific embodiment, the “variable regions” and “CDRs” refer to variable regions and CDRs defined by the IMGT approach.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

According to specific embodiments the antibody is a recombinant antibody.

As used herein, the term “recombinant antibody” refers an antibody produced by recombinant DNA techniques, i.e., produced from cells transformed by an exogenous DNA construct encoding the antibody.

According to specific embodiments the antibody is a monoclonal antibody.

In cases where target antigens are too small to elicit an adequate immunogenic response when generating antibodies in vivo, such antigens (referred to as “haptens”) can be coupled to antigenically neutral carriers such as keyhole limpet hemocyanin (KLH) or serum albumin (e.g., bovine serum albumin (BSA)) carriers (see, for example, U.S. Pat. Nos. 5,189,178 and 5,239,078). Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Illinois, USA. The resulting immunogenic complex can then be injected into suitable mammalian subjects such as mice, rabbits, and others. Suitable protocols involve repeated injection of the immunogen in the presence of adjuvants according to a schedule designed to boost production of antibodies in the serum. The titers of the immune serum can readily be measured using immunoassay procedures which are well known in the art.

The antisera obtained can be used directly or monoclonal antibodies may be obtained, as described hereinabove. Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Thus, antibodies of the present invention are preferably at least bivalent (e.g., of the IgG subtype) or more (e.g., of the IgM subtype). It will be appreciated that monovalent antibodies may be used however measures should be taken to assemble these to larger complexes such as by using secondary antibodies (or using other cross-linkers which are well known in the art). According to specific embodiments the antibodies are from IgG1 subtype.

According to specific embodiments antibody is a humanized or partially humanized antibody.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51(1996); Neuberger, Nature Biotechnology 14: 826 (1996); and Lonberg and Huszar, Intern. Rev. Immunol. 13: 65-93 (1995).

The antibodies can be mono-specific (i.e., binding a distinct antigen) or multi-specific (i.e. binding at least two different epitopes, e.g., bi-specific or tri-specific).

According to specific embodiments, the antibody is a mono-specific antibody.

According to specific embodiments, the antibody is bi-specific antibody.

According to specific embodiments, the antibody is a tri-specific antibody.

According to other specific embodiments, the antibody is a multi-specific antibody.

### AXL Inhibitors

As used herein, the term “AXL inhibitor” refers to an agent, compound or substance which is capable of inhibiting the function or activity of the AXL receptor, for example, by binding or not to the AXL receptor. Particularly, the AXL inhibitor as used herein can be a small molecule organic compound, an antibody and a polynucleotide. A suitable AXL inhibitor according to the present invention can be identified by persons skilled in the art using various known methods, for example, by its ability to bind to the AXL receptor and inhibit the kinase activity or by its ability to block or reduce the gene expression of the AXL receptor. Specifically, the AXL inhibitor of the invention blocks the phosphorylation of AXL at amino acid T702 and/or Y779. In some embodiments, the AXL kinase activity or expression level is reduced, by about 10% less, about 20% less, about 30% less, about 40% less, about 50% less, about 60% less, about 70% less, about 80% less, about 90% less, or completely blocked by an AXL inhibitor of the present invention, as compared with a control AXL not exposed to the AXL inhibitor.

In some embodiments, the AXL inhibitor of the invention is an AXL antagonist which is capable of specifically binding to the AXL receptor and inactivating, fully or partially, the AXL activity. In certain embodiments, an AXL antagonist of the invention is a small organic molecule. As used herein, the term “small organic molecule” is recognized in the art and refers to a molecule of a size comparable to those organic molecules generally used in pharmaceuticals, which does not include biological macromolecules such as proteins or nucleic acids. Preferred small organic molecules are characterized as having a size less than 10,000 Da, more preferably less than 5,000 Da, even more preferably less than 2,000 Da, and most preferably less than 1,000 Da.

In other embodiments, the AXL inhibitor of the invention is an antagonist antibody that specifically binds to AXL and inactivating, fully or partially, the AXL activity.

In one embodiment, the AXL inhibitor acts by enhancing endocytosis of AXL or by means of recruiting immune-effector cells, such as NK cells.

According to a particular embodiment, the AXL inhibitor is a small molecule inhibitor. Examples of such inhibitors include, but are not limited to R428, bemcentinib, YW327.652, GL2I.T, TP-0903, LY2801653, amuvatinib, bosutinib, MGCD 265, ASP2215, cabozantinib, foretinib, SGI-7079, MGCD516, ASLAN002, and gilteritinib.

According to a particular embodiment, the AXL inhibitor is bemcetinib.

According to a particular embodiment, the AXL inhibitor is an antibody, as defined herein above.

The antibodies can be selected from pre-existing antibodies (e.g., publicly available hybridomas or recombinant antibody libraries, further described herein below) or from newly generated antibodies produced according to methods which are well-known in the art and further described herein.

Antibodies and methods of generating same are described at length in the following sections.

Additional examples of AXL specific antibodies are disclosed in US Patent Application No. 20210070869, 20190275149 and 20190352407, the contents of which are incorporated herein by reference.

For example, the light chain of a particular antibody which is directed to AXL may be encoded by the nucleic acid sequence as set forth in SEQ ID NO: 7.

CDR1 of the light chain:
(SEQ ID NO: 1)
QNVDTN
CDR2 of the light chain:
SAF
CDR3 of the light chain:
(SEQ ID NO: 3)
QQYNNYPYT

An exemplary amino acid sequence of the light chain is set forth in SEQ ID NO: 8.

For example, the heavy chain of antibody which is directed to AXL may be encoded by the nucleic acid sequence as set forth in SEQ ID NO: 9.

CDR1 of the heavy chain:
(SEQ ID NO: 4)
GFTFSTYG
CDR2 of the heavy chain:
(SEQ ID NO: 5)
INGNGGSA
CDR3 of the heavy chain:
(SEQ ID NO: 6)
VRERDYYGNSRYFDV

An exemplary amino acid sequence of the heavy chain is set forth in SEQ ID NO: 10.

It will be appreciated that when the AXL inhibitor is an antibody, the present inventors also contemplate use of bispecific antibodies, wherein the first arm of the bispecific antibody targets the EGFR and the second arm of the antibody targets the AXL.

As used herein the term “cancer” refers to a tumoral disease which depends on ErbB (activity and/or expression) (e.g. EGFR) for onset and/or progression. Thus, the cancer cells express an ErbB polypeptide which facilitates disease progression.

Examples of cancer which can be treated in accordance with the present teachings include, but are not limited to invasive breast carcinoma, adenocarcinoma, lung cancer (non-small cell, squamous cell carcinoma, adenocarcinoma, and large cell lung cancer), liver cancer, colorectal cancer, brain, head and neck cancer (e.g., neuro/glioblastoma), breast cancer, ovarian cancer, transitional cell carcinoma of the bladder, prostate cancer, oral squamous cell carcinoma, bone sarcoma, adrenocortical cancer, gastrointestinal tumors including colorectal cancer, biliary tract cancer such as gallbladder carcinoma (GBC), bladder cancer, esophageal cancer, gastric cancer, cervical cancer, salivary gland cancer, diarrhea benign neoplasm, ductal carcinoma in situ, paronychia, cholangiocarcinoma, kidney cancer, pancreatic cancer, medulloblastoma, glioblastoma, luminal, HER2-positive and triple negative mammary tumors and viral leukemia.

According to a specific embodiment the cancer is lung cancer.

According to specific embodiments the lung cancer is non-small cell lung cancer (NSCLC).

As used herein, the phrase “resistance to a tyrosine kinase inhibitor (TKI)” refers to non-responsiveness to TKI treatment as may be manifested by tumor size, in-vitro activity assays and/or patient survival.

According to a specific embodiment, resistance refers to no amelioration in disease symptoms or progression according to a regulatory agency guidelines (e.g., FDA) for the specific TKI used. Resistance to treatment can be primary resistance or acquired resistance.

According to specific embodiments the resistance is an acquired resistance.

As used herein the term “acquired resistance” refers to progression of resistance following initial positive response to therapy.

The main known molecular mechanism of acquired resistance to TKIs include mutations in the e.g. EGFR kinase domain, including T790M; gene amplification, such as MET, over-expression of RTK ligands that mediates uncontrolled tumor cell activation; modification of signaling pathways, such as PTEN instability that mediates constitutive Akt activation; and increased efflux or decreased influx of TKIs from the cancer cell, mediated by membrane transporters such as MDR1 or hOCT1 [see e.g. Chen and Fu, Acta Pharmaceutica Sinica B, (2011) 1(4): 197-207].

Thus, according to a specific embodiment, the cancer cells express an ErbB receptor (e.g. EGFR) having a mutation in a kinase domain of the receptor.

According to another embodiment, the cancer is resistant to a TKI when provided as a monotherapy

As used herein, the phrase “resistance to a tyrosine kinase inhibitor (TKI)” refers to non-responsiveness to TKI treatment (when provided as a monotherapy) as may be manifested by tumor size, in-vitro activity assays and/or patient survival.

According to a specific embodiment, resistance refers to no amelioration in disease symptoms or progression according to a regulatory agency guidelines (e.g., FDA) for the specific TKI used. Resistance to treatment can be primary resistance or acquired resistance.

According to specific embodiments the resistance is an acquired resistance.

As used herein the term “acquired resistance” refers to progression of resistance following initial positive response to therapy.

According to specific embodiments the patient further exhibits resistance to an anti-ErbB monoclonal such as but not limited to anti-EGFR (e.g. cetuximab).

The main known molecular mechanism of acquired resistance to TKIs include mutations in the e.g. EGFR kinase domain, including T790M; gene amplification, such as MET, leading to overproduction of the TK; over-expression of RTK ligands that mediates uncontrolled tumor cells activation; modification of signaling pathways, such as PTEN instability that mediates constitutive Akt activation; and increased efflux or decreased influx of TKIs from the cancer cell, mediated by membrane transporters such as MDR1 or hOCT1 [see e.g. Chen and Fu, Acta Pharmaceutica Sinica B, (2011) 1(4): 197-207].

Thus, according to a specific embodiment, the cancer cells express an EGF receptor having a mutation in a kinase domain of said receptor.

Methods of analyzing sequence alterations such as in the kinase domain of an EGFR are well known in the art, basically including analysis (e.g., by PCR and sequencing) of genomic DNA, or cDNA encoding the ErbB using a biological sample obtained from the subject exhibiting the resistance (e.g., biopsy). Analysis at the polypeptide level can also be done such as using antibodies which specifically recognize the mutated form of the protein and not the wild-type form. Analysis at the protein level can also be done by an activity assay as further described hereinbelow.

Such biological samples include, but are not limited to, body fluids such as whole blood, serum, plasma, cerebrospinal fluid, urine, lymph fluids, and various external secretions of the respiratory, intestinal and genitourinary tracts, tears, saliva, milk as well as white blood cells, malignant tissues, amniotic fluid and chorionic villi.

According to one embodiment the sample comprises a fluid, such as for example, blood, plasma, saliva etc.

The sample may comprise cells including, but not limited to blood cells, bone marrow cells, pancreatic cells, lung cells, hepatic cells, spleen cells, kidney cells, cardiac cells, ovarian cells, breast tissue cells, skin cells (e.g., epithelial cells, fibroblasts, keratinocytes), lymph node cells.

According to a particular embodiment the cells comprise cancer cells. Such cells can be obtained using methods known in the art, including, but not limited to, fine needle biopsy, needle biopsy, core needle biopsy and surgical biopsy (e.g., lung biopsy), buccal smear and lavage.

Mutations in the kinase domain of the receptor may alter the kinase activity.

According to specific embodiments, the mutation does not substantially affect a kinase activity of the EGFR.

As used herein, the term “substantially affect” refers to an un-altered kinase activity (+/−10%, or 20%) in the presence of absence of the mutation.

Determining the kinase activity can be achieved using methods well known in the art, such as Western-blot and in-vitro kinase assay.

Non limiting examples of mutations in a kinase domain of EGFR include: G719C, G719S, L858R, L861Q, T790M and an exon 20 insertion.

According to specific embodiments the mutation comprises the T790M mutation.

As used herein, the term “T790M” refers to a substitution of Threonine to Methionine at position 790 (T790M) in the EGFR kinase domain. This substitution was shown to preserve (i.e., not substantially affect) the kinase activity of the receptor.

The agents may be formulated each in a different formulation, two in one formulation and the other one in a separate formulation, or all in the same formulation i.e.: anti-EGFR, TKI and AXL inhibitor; anti-EGFR+TKI and AXL inhibitor; anti-EGFR+AXL inhibitor and TKI; or anti-EGFR+TKI+AXL inhibitor.

According to specific embodiments, the active ingredients are in a co-formulation.

According to other specific embodiments, the active ingredients are in separate formulations.

The antibodies and/or TKIs of the present invention can also be attached to a cytotoxic agent or provided together with a cytotoxic agent.

Thus, for example, the antibodies and/or TKIs of the present invention can be administered along with analgesics, chemotherapeutic agents (e.g., anthracyclins), radiotherapeutic agents, hormonal therapy and other treatment regimens (e.g., surgery) which are well known in the art.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the antibodies accountable for the intended biological effect.

Hereinafter, the phrases “physiologically acceptable carrier” and “pharmaceutically acceptable carrier”, which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington' s Pharmaceutical Sciences,” Mack Publishing Co., Easton, PA, which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., a nucleic acid construct) effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Since administration of the triplet combination is expected to produce improved results over the administration of single agents, the therapeutically effective dose of each of the agents in the combined treatment may be for example less than 50%, 40%, 30%, 20% or even less than 10% the of the FDA approved dose.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl E. et al. (1975), “The Pharmacological Basis of Therapeutics,” Ch. 1, p.1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations (e.g., weekly or bi-weekly administrations), with course of treatment lasting from several days to several weeks, or until cure is effected or diminution of the disease state is achieved.

According to specific embodiments the administering comprises multiple administrations.

According to specific embodiments the multiple administrations comprise bi-weekly administrations.

The amount of a composition to be administered will, of course, be dependent on the subject being treated, the severity of the affliction, the manner of administration, the judgment of the prescribing physician, etc.

Typically used models for analyzing the effect of the agents described herein on tumors are provided infra.

An animal lung tumor model expressing a T790M mutated EGFR is described e.g. in Regales et al. PLoS ONE (2007) 2:e810 and Politi et al. Genes Dev. (2006) 20:1496-1510.

Suitable cells for use in animal models and in vitro analyses include but are not limited to H1975, PC9ER, H820, HCC827 and H1650.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. Each of the agents may be provided in separate packaged or may be packaged in a single packaging.

The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques.

### Materials And Methods

Cell cultures and reagents: HEK-293T cells were cultured in DME medium supplemented with fetal bovine serum (10%; FBS). PC9, H1299 and H1975 cells were grown in RPMI medium supplemented by 10% FBS. The cells were procured from ATCC and checked for mycoplasma. Erlotinib was from LC Laboratories, afatinib and imatinib from MedChem Express, and osimertinib was a gift from AstraZeneca. All TKIs were dissolved in DMSO at a stock concentration of 10 mM. Cetuximab and trastuzumab were obtained from Merck and Roche, respectively. Oligonucleotide primers were obtained from Sigma.

Generation of AXL overexpressing, luciferase-tagged PC9 cells: Lentiviral particles were produced in HEK293FT cells (Thermo Fischer Scientific, Germany) by co-transfecting lentiviral expression vectors containing the coding region of either AXL (pLX304-AXL) or red firely luciferase (rwpLX305_IRES_Puro-RedFF), together with 2^{nd} generation viral packaging plasmids (VSV.G from Addgene #14888) and psPAX2 (Addgene #12260). Forty-eight hours after transfection, virus-containing supernatant was removed and cleared by centrifugation. The supernatant was passed through a 0.45 μm filter. PC9 cells were transduced with lentiviral particles at 75% confluency, in the presence of 10 μg/ml polybrene (Merck, Germany). Twenty-four hours after transduction, virus-containing medium was replaced with selection medium for the respective expression constructs (blasticydin for pLX304-AXL and puromycin for rwpLX305_IRES_Puro-RedFF) to establish the overexpressing PC9 cell lines.

In vitro knockout of the genes encoding for AXL and RAD18: The CRISPR-CAS9 system was used to create a double-stranded break next to the Protospacer Adjacent Motif (PAM). The target site was selected using the ENSEMBL database in a way that targeted the transcripts of AXL or RAD18. The selected targets, 21 bp long, included the PAM sequences in exon 5 and in exon 2, respectively. The sequences were filtered to minimize off-target cross-reactivity. The respective sequences were cloned in pSpCas9(BB)-2A-GFP. The resulting plasmids were transfected into PC9 cells using Lipofectamine 2000 (from Invitrogen). GFP-positive sorting was performed to obtain single cell clones.

Generation of anti-AXL mAbs: Mouse immunization, fusion between myeloma cells and splenocytes, and the subsequent hybridoma subcloning were performed as described (52). A fusion protein combining the extracellular domain of AXL and the Fc domain of a human IgG1 was constructed and used for immunization of mice. For selection of positive hybridomas, supernatants were incubated with protein G for 1 hour and the antibody-bead complexes were washed prior to adding A549 cell lysate. Sixty-minutes later, the immunoprecipitates were washed, resolved using electrophoresis and transfer to a nitrocellulose membranes. Membranes were blocked with TBS-T (tris-buffered saline containing 0.1% Tween-20) containing 1% low-fat milk Membranes were blotted overnight with a primary antibody, washed three times with TBS-T, incubated for 30 minutes with a secondary antibody linked to horseradish peroxidase, and washed once again with TBS-T. Immunoreactive bands were detected using the ECL reagent (Biorad).

Drug tolerant persister assays: Drug-sensitive PC9 cells were treated with the relevant TKI, at concentrations exceeding 100 times the established IC50 values, for three rounds, with each treatment lasting 72 hours. Viable cells remaining attached on the dish at the end of the third round of drug treatment were considered to be DTPs, hence they were collected for analysis.

Generation of pDRGFP expressing cells: The PC9 and its derivatives, AXL-overexpression or AXL-knockout cells, were seeded at 25×10⁴ cells/well in 6-well plastic culture plates. On the following day, cells were transfected with pDRGFP plasmid (Addgene) using Lipofectamine 2000. The plasmid is composed of two differentially mutated GFP (green fluorescent protein) genes oriented as direct repeats and separated by a drug selection marker (25). One of the GFP genes is mutated to contain the recognition site for the SceI endonuclease and, as a result, will undergo a DSB when Sce-I is ectopically expressed. A homologous recombination event between the two GFP genes results in the expression of intact GFP protein. Twenty-four hours after infection, puromycin (5 μg) was used to select stably infected cells.

Homologous recombination assays: The pDRGFP-expressing cells were seeded at 25×10⁴ cells/well in 6-well culture plates. On the following day, cells were transfected with the Sce-I -expressing plasmid (pCBASce-I; Addgene) (25) Immediately after transfection, cells were treated with erlotinib or osimerinib, as indicated in the legends to figures. Twenty-four hours after treatment, cells were harvested and analyzed using flow cytometry. The relative HR capacity was determined by dividing the percentage of GFP-positive cells in the Sce-I transfected cultures by the percentage of GFP signal in mock control.

Whole exome sequencing: Genomic DNA was isolated using PureLink™ Genomic DNA Mini Kit (Cat# K182002). The qualtiy of the DNA was checked by agarose gel ecltrophoresis and Qubit dsDNA HS Assay kit (Thermo Fisher Scientific). Exome sequencing libraries were prepared according to the manufacturer's instructions (Twist Human core exome kit+RefSeq Panel). Input gDNA (50 ng) from each sample was processed for enzymatic fragmentation, subsequent end repair, and dA-tailing to generate dA-tailed DNA fragments. Twist Universal Adapters were ligated to the dA-tailed DNA fragments, and purified to generate gDNA libraries ready for index introduction through amplification. A gDNA library adapted with Twist CDI primers was amplified and purified. The quality of the amplified library was confirmed using capillary electrophoresis (Bioanalyzer, Agilent). Prior to hybridization, the volume of each library was adjusted using the respective concentration of each Amplified Indexed Library. Ten libraries were multiplexed by 150 ng to give a total mass of 1500 ng. The pre-hybridization solution containing library, probes, RefSeq Panel and blockers was dried using a SpeedVac system and low heat. 0.020 ml of the fast hybridization mix was added to the dried pre-hybridization solution, and then 0.030 ml hybridization enhancer was added on top of the pre-hybridization solution. The mixture was incubated for 5 min at 95° C. and later heated for 2 hours at 60° C. in a thermal cycler (Fast Hybridization). Streptavidin Binding Beads (100 μl) were washed three times with 0.2 ml Fast Binding Buffer and re-suspended in 0.2 ml binding buffer. The hybridization mixture was added to the bead suspension and incubated for 30 min at RT with mixing. The beads were washed once, preheated in Fast Wash Buffer 1 for 5 min at 70° C., and three times with 0.2 ml of Wash Buffer2 for 5min at 48° C. DNA was eluted with 0.045 ml nuclease-free water (Streptavidin Binding Bead slurry). The captured library was amplified using PCR to enrich for fragments. The quality of the amplified libraries was verified by means of automated electrophoresis (Tapestation, Agilent). Exome sequencing was performed using an Illumina NovaSeq6000 system.

Alignment and variant calling: Sequence QC was done through FastqQC 0.11.5 (Andrews 2015). Reads were mapped to human reference genome sequence (hg19; NCBI GRCh37) using bwa 0.7.12 (H. Li and Durbin 2009). BAM files were realigned with the Genome Analysis Toolkit 3.5 (McKenna et al. 2010) (GATK) IndelRealigner, and base quality scores were recalibrated by the GATK base quality recalibration tool. Variants were called with GATK's HaplotypeCaller tool 3.5. To filter potential errors, GATK's Variant Quality Score Recalibration (VQSR) was conducted based on hapmap 3.3, NCBI Variation Database (dbSNP138), 1000 genomes and Omni 2.5M SNP chip array. Then, the variants' functional information was annotated using SnpEff 4.2(GRCh 37.75) (Cingolani et al. 2012).

Comet assays: Cells (2.5×10⁵) were plated in 6-well plates and incubated overnight for attachment. On the following day, cells were treated with drugs and following additional 24 hours we performed the comet assay using a kit (from Abcam) and the alkaline electrophoresis buffer. Comets stained with FITC were viewed using an Olympus XM10 epifluorescence microscope and analyzed with CASPlab software.

Digital PCR: Mutation analysis specific for the T790M alteration was performed using the Bio-Rad droplet digital PCR (ddPCR) platform. Genomic DNA was isolated from cell lines using Pure Link Genomic DNA mini kit. Stocks (10 ng/μl ) of each DNA sample were used for the experiment. PC9ER cells that harbor T790M mutation were used as positive control. Cycling conditions were as follows: 95° C. for 10 mins (1 cycle), 40 cycles of 94° C. for 30 seconds and 55° C. for 1 min, followed by 98° C. for 10 mins and hold at 4° C. The count of partitions showing positive amplification was obtained using the QuantaSoft Software.

Reverse phase protein array profiling: Lysates were adjusted to a total protein concentration of 2 μg/μL, mixed with 4×SDS sample buffer (10% glycerol, 4% SDS, 10 mM DTT, 125 mM Tris-HCl, pH 6.8) and denatured at 95° C. for 5 min. Lysates and dilution series of each cell line, serving as controls, were spotted as technical triplicates on nitrocellulose-coated glass slides (Grace-Biolabs, Bend, OR) using an Aushon 2470 contact spotter (Aushon BioSystems, Billerica, MA). Post spotting, slides were incubated with blocking buffer (Rockland Immunochemicals, Gilbertsville, PA) containing 5 mM NaF and 1 mM Na₃VO₄ for 2 hours at room temperature, prior to incubation with target-specific primary antibodies at 4° C., overnight. Primary antibodies were detected using Alexa Fluor 680-coupled goat anti-mouse IgG or anti-rabbit IgG at 1:8000 dilutions (Life Technologies, Darmstadt, Germany). In addition, representative slides were stained for total protein quantification using Fast Green FCF protein dye. TIFF images of all slides were obtained at an excitation wavelength of 685 nm and at a resolution of 21 μm using an Odyssey Scanner (LI-COR, St. Lincoln, NE). Signal intensities of individual spots were quantified using GenePixPro 7.0 (Molecular Service, Sunnyvale, CA). Data preprocessing, merging of technical triplicates, background correction, and quality control were performed using the RPPanalyzer Rpackage.

ROS production assay: Cells were seeded on 96-well white-walled plates (10×10³ cells/well) and incubated overnight for attachment. The following day, cells were treated and after 48 hours ROS were measured by using the ROS-Glo™ H₂O₂ Assay kit (Promega) according to the manufacturer's protocol. Luminescence was measured using a plate-reading luminometer (TECAN, Infinite® 200 PRO Nano Quant) and the resulting data were normalized to untreated cells at each time point.

Proximity ligation assays: Cells plated on glass coverslips were fixed, permeabilized, blocked, and incubated with the indicated antibodies. PLA was performed using the DuoLink In Situ PLA Detection Kit (Sigma). Hybridization with PLA probes, ligation, and amplification of the signal were performed according to the manufacturer's instructions. Images were taken using Zeiss LSM800 confocal microscope at objective ×63. PLA signal was quantified per cell using the ImageJ software. PLA and depicted in red, whereas Phalloidin was depicted in green. Experiments were done in triplicates, and 20 cells were used per condition per experiment.

Immunoprecipitation assays: To pull down endogenous AXL and RAD18 expressed in PC9 cells, we incubated protein A beads (Sigma) with anti-AXL and anti-RAD18 antibodies (Cell Signaling Technology) at 4° C., for 1 hour. Beads were washed thrice with HNTG buffer (25 mM HEPES, pH 7.5, 150 mM NaCl, 10% glycerol, 0.1% Triton X-100). Cell lysates were added to the complex of beads and antibody, and incubated further at 4° C. Beads and the complexes were washed three time with HNTG buffer. Next, the immunoprecipitates were resolved using electrophoresis, followed by transfer to a nitrocellulose membrane. Membranes were blocked with TBS-T (tris-buffered saline containing Tween-20) containing 1% low-fat milk, blotted overnight with a primary antibody, washed three times with TBS-T and incubated for 30 minutes with a secondary antibody linked to horseradish peroxidase prior to washing, once again, with TBS-T. Immunoreactive bands were detected using the ECL reagent (Biorad).

Metabolite extraction and LC-MS polar metabolites analysis: Polar substance extraction and analysis was performed as previously described (53) with some modifications: cell extracts were mixed with 1 ml of a pre-cooled (−20° C.) homogenous methanol: methyl-tertbutyl-ether (TMBE) 1:3 (v/v) mixture. The tubes were vortexed and then sonicated for 30 minutes in an ice-cold sonication bath. Then, UPLC grade water: methanol (3:1, v/v) solution (0.5 ml) containing internal standards (a mixture of 13C and 15N labeled amino acids, from Sigma) was added to the tubes followed by centrifugation. The polar phase was re-extracted as described above. The lower (polar) phase used for polar metabolite analysis was stored at −80° C. until analysis. Finally, the polar phase samples were lyophilized and the pellets were dissolved using 0.15 ml water:methanol (1:1) and centrifuged twice prior to loading onto the LC-MS system. Metabolic profiling was performed using Acquity I class UPLC System combined with a mass spectrometer (Thermo Exactive Plus Orbitrap), which was operated in a negative ionization mode. LC separation was performed using SeQuant Zic-pHilic (150 mm×2.1 mm) column with the SeQuant guard column (20 mm×2.1 mm; from Merck). The mobile phases employed were 20 mM ammonium carbonate with 0.1% ammonia hydroxide in water: acetonitrile (80:20, v/v; Mobile phase A) and acetonitrile as Mobile phase B. The flow rate was kept at 0.2 ml per minutes and the following gradient: 0-2 min 75% of B, 14 min 25% of B, 18 min 25% of B, 19 min 75% of B, for 4 min For data processing we used TraceFinder (from Thermo Fisher), which detected compounds on the basis of accurate mass, retention time, isotope pattern, fragments and an in-house mass spectra library.

Beta gal staining: Cells were seeded on 6-well plates and allowed to reach 70% confluency. The cells were fixed with 1% glutaraldehyde followed by washing with PBS and staining with β-galactosidase (β-Gal) for 6 hours at 37° C. The reaction was terminated by replacing the reaction mixture with PBS. Images were captured using Olympus SZX16 microscope.

Cell migration and invasion assays: Cells were plated in the upper compartment of 24-well transwell trays (Corning, Acton, MA). Thereafter, the medium in the lower compartment was supplemented with the indicated agents and cells were allowed to migrate for 16 hours at 37° C. through the intervening nitrocellulose membrane (8 μm pore size). The filter was later removed and attached cells were fixed for 15 minutes in saline containing paraformaldehyde (4%). Staining with crystal violet followed this step. Cells growing on the upper side of the filter were scraped using a cotton swab; cells located on the bottom side were photographed and counted. Similarly, cell invasion assays were performed using BioCoat Matrigel Invasion Chambers (BD Bioscience, Franklin Lakes, NJ).

Thymidine incorporation assays: Cells were plated onto 24-well plates at a density of 5×10⁴ cells/well, followed by plasmid transfection. Sixteen hours later, cells were incubated with fresh serum-free medium containing ³H-thymidine (1 μCi). After 48 hours, the reaction was terminated by the addition of ice-cold trichloroacetic acid (5%; TCA). Five minutes later, cells were solubilized at 37° C. in 1N NaOH (for 10 minutes) followed by 1N HCL. Quadruplicate samples were collected into scintillation vials. Radioactivity was determined in a scintillation counter.

Cell lysis and immunoblotting: Cell lysates were collected in a lysis buffer (50 mM HEPES, pH 7.5, 10% glycerol, 150 mM NaCl, 1% Triton X-100, 1 mM EDTA, 1 mM EGTA, 10 mM NaF and 30 mM b-glycerol phosphate). Cleared cell lysates were collected using centrifugation (12,000 rpm for 20 minutes) and further resolved using electrophoresis followed by transfer to nitrocellulose membranes. Membranes were blocked with TBS-T (tris-buffered saline containing Tween-20) containing 1% low-fat milk, blotted overnight with a primary antibody, washed three times with TBS-T, incubated for 30 minutes with a secondary antibody linked to horseradish peroxidase, and washed once again with TBS-T Immunoreactive bands were detected using the ECL reagent (Biorad).

RNA isolation and real-time PCR analysis: Total RNA was extracted using the Perfect Pure RNA Cultured Cell Kit (5-prime, Hamburg) according to the manufacturer's instructions. Total RNA quantity and quality were determined using the Nano Drop ND-1000 spectrophotometer (Thermo Fischer Scientific, Waltham, MA). Complementary DNA was synthesized using the High Capacity Reverse Transcription kit (Applied Biosystems, Life Technologies, Carlsbad, CA, USA). Real-time qPCR analysis was performed with SYBR Green (Applied Biosystems) and specific primers on the Step One Plus Real-Time PCR system (Applied Biosystems). Signals (cT) were normalized to actin.

Luciferase-reporter assays: Cells were co-transfected with a luciferase reporter plasmid, along with pGL3-Control (Promega, Madison, WI). Luciferase activity was determined using the dual-luciferase reporter assay system (Promega). Firefly luciferase luminescence values were normalized to Renilla luminescence.

Nucleotide sequencing of RNA: RNA was isolated using Dynabeads mRNA Direct Kit (Thermo Fisher Scientific). NGS libraries were prepared using a modified version of Transeq, as described (54). In brief, RNA was barcoded and reverse-transcribed using poly-T primers, followed by addition of exonuclease to remove excess RT-PCR primers. Next, the single-stranded cDNA was converted to a double-stranded DNA. The template DNA was then removed using DNase and the generated RNA was fragmented and ligated to barcoded Illumina adapters. Reverse transcription of this ligation product was performed using primers specific for the Illumina adapters, and libraries of the resulting cDNA were generated and enriched by performing 12-15 PCR cycles. RNA-seq libraries (pooled at equimolar concentrations) were sequenced on an Illumina NextSeq 500 at a median sequencing depth of 10 million reads per sample. Sequences were mapped to the human genome and filtered. Quality checks, pre-processing, alignment and differential expression analysis were performed using the “User-friendly Transcriptome Analysis Pipeline” (UTAP) (55). Differential expression analysis was performed using DESeq2 (56). Genes were considered to be differentially expressed if their p-value was smaller or equal to 5e-06 and Log Fold Change threshold ±1. The tool “Enrichr” (57, 58) was used to perform pathway enrichment analysis. A graph with the most significantly enriched pathways from the “NCATS BioPlanet” integrative platform (59) was prepared. All plots and graphs related to RNA-Seq data analysis were prepared using R version 3.6.2.

Immunofluorescence analyses: Immunofluorescence was performed on cells grown on sterile coverslips. Briefly, following 24 hours of treatment, cells were washed in saline containing Tween 20 (0.01%; w/v). Thereafter, cells were fixed with 4% formaldehyde in saline (overnight at 4° C.). On the next day, cells were blocked for 30 min with fetal bovine serum (2%). Next, they were incubated overnight at 4° C. with anti-receptor antibodies (1:50 dilution). Thereafter, cells were washed thrice, followed by the CY3-conjugated secondary antibody (45 min in dark), counterstained with DAPI and mounted on slides for image capturing using confocal microscope (40× magnification). Images were processed using the Zeiss ZEN2011 software.

RNA interference: Cells were transfected with On-Target-specific siRNA oligonucleotides, as well as scrambled siRNAs (siCTRL), which were purchased from Dharmacon (GE Healthcare). For all transfections, cells were seeded in 6-well plates and siRNA oligonucleotide transfection was performed using Oligofectamine (Invitrogen) according to the manufacturer's instructions.

Cell cycle analysis: Cells were incubated for 60 minutes with bromodeoxyuridine (BrdU; 10 μM) and then washed, harvested and fixed in ethanol (at 4° C.). Thereafter, cells were incubated in a denaturation solution (2N HCl, 0.5% Triton-X100; 30 min), followed by a neutralization solution (0.1 M sodium borate, pH 8.5; 30 min). BrdU that incorporated into newly synthetized DNA was then assayed using an FITC-conjugated anti-BrdU antibody. Total DNA content was determined by using a propidium iodide (PI) solution supplemented with RNase A. Cell cycle distribution was detected using flow cytometry. Further analysis was performed using the FlowJo software v10.2 (Tree Star).

Receptor expression assays: To evaluate surface receptor levels, cells were mildly trypsinized and washed twice in saline containing albumin (1% w/v). Thereafter, cells were incubated for 30 minutes at 4° C. using primary antibodies conjugated to specific fluorophores (AXL-APC and EGFR-FITC). Fluorescence intensity was determined using BD FACS Aria Fusion flow cytometer.

Cell viability assays: Cell viability was assessed by using MTT (3-(4,5-dimethylthiazol-2-yl)-2,5-diphenyltetrazolium bromide). PC9 cells (5×10³/well) were seeded in 96-well plates. On the next day, cells were treated for 72 hours with the indicated drugs. Afterwards, cells were incubated for 3 hours at 37° C. with the MTT solution (0.5 mg/ml). The formazan crystals formed by metabolically active cells were dissolved in DMSO and the absorbance was read at 570 nm.

Apoptosis assays: Cells were seeded in 10-cm dishes. On the next day, complete media were replaced with media containing foetal bovine serum (1%) and cells were treated for 48 hours with the indicated drugs. Apoptosis was assessed using flow cytometry and the FITC Annexin V Apoptosis Detection Kit with 7-AAD (from BioLegend). The analysis was performed on the BD LSR II cytometer (BD Biosciences).

Animal experiments: All animal studies were approved by our institutional board and they were performed in accordance with the guidelines of the Institutional Animal Care and Use Committee (IACUC). PC9 cells (2.5×10⁶ per mouse, in 0.1 ml) were subcutaneously injected in the right flanks of CD1 nude mice (5-6 weeks old). Once tumors reached a volume of approximatively 500 mm³, mice were randomized into different groups and treated as indicated. TKIs were daily administered using oral gavage. Antibodies were administered twice a week using intraperitoneal injection at a final dose of 200 μg/mouse/injection. Tumor volume was estimated using vernier caliper measurements of the longest axis, a/mm, and the perpendicular axis, b/mm Volumes were calculated in accordance with the equation V=(4 π/3)×(a/2)²×(b/2). Animals were euthanized when tumor size reached 1,400-1,500 mm³. For metastasis assays, NOG mice were injected intravenously (2×10⁵ per mouse). Mice were sacrificed four weeks after injection, and the lungs were analyzed. Lung images were acquired, and the numbers of micro-metastases or nodules were quantified.

Statistical analysis: Microsoft Excel, GraphPad Prism (version 8.0.2) and R (version 3.6.2) software packages were used to analyze the data. Statistical analyses were performed using Fisher's exact test and one or two-way ANOVA with the Dunnett's or Tukey's test (*, p≤0.05; **, p≤0.01; ***. p≤0.001; ****, p≤0.0001). Flow cytometry analysis was performed on a BD FACSAria Fusion Instrument. Further analysis was performed using the FlowJo software v10.2 (Tree Star). Staining intensity was determined using ImageJ. Sample numbers and other information (mean ±SEM or SD, number of replicates and specific statistical tests) are indicated in the main text or in figure legends.

### Results

### TKI-Induced Activation of AXL is Essential for Emergence of Resistance

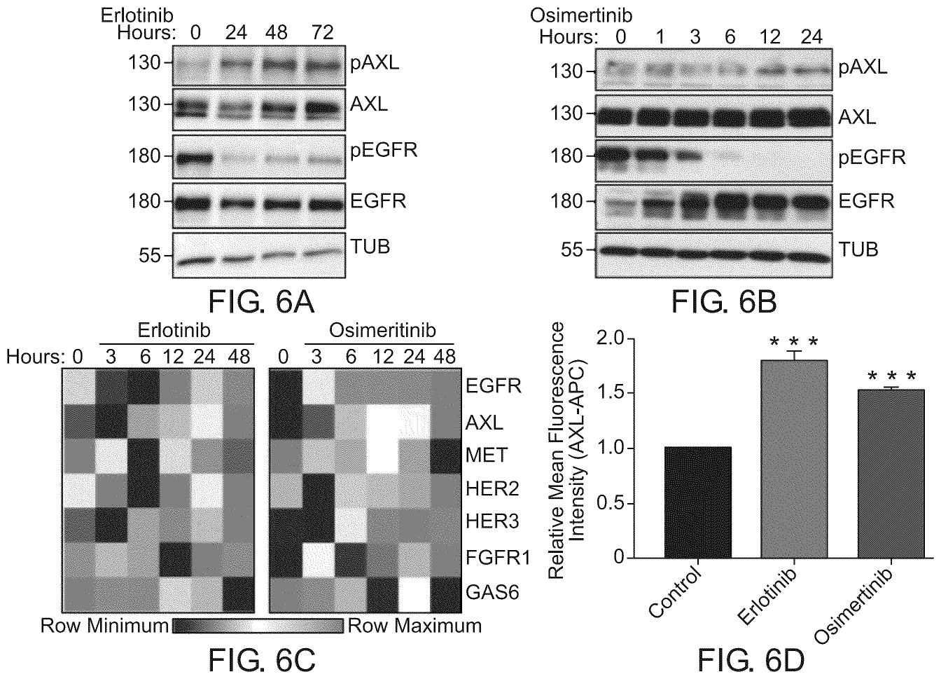

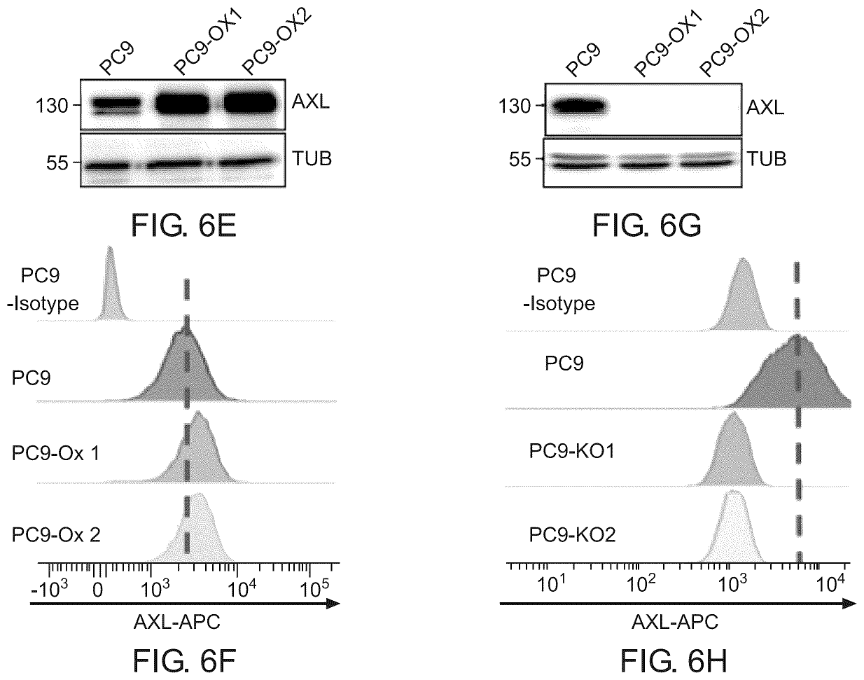

Upon long-term treatment of PC9 cells (E746_A750 del-EGFR) with TKIs, EGFR phosphorylation was inhibited, but AXL underwent activation, rather than inhibition (FIGS. 6A and 6B). Reverse-phase protein arrays (RPPA) confirmed up-regulation of AXL and other RTKs (FIG. 6C). Flow cytometry indicated that the newly formed AXL molecules were delivered to the cell surface (FIG. 6D). While similar observations have previously been reported (15, 17), the contribution of adaptive AXL activation to drug resistance has remained incompletely understood. Hence, PC9 cells were implanted in mice and once tumors became palpable, daily TKI treatments were commenced (FIG. 1A). In analogy to clinical scenarios, all tumors initially regressed but later exhibited robust relapses. Analysis of tumor extracts (FIG. 1B) detected up-regulation of AXL in TKI-treated tumors, along with sporadic ERK inactivation and AKT activation. To address significance of adaptive AXL up-regulation, both gain- and loss-of-function strategies were undertaken. Two clones of AXL-overexpressing (OX) PC9 cells were established (FIGS. 6E and 6F) Similarly, CRISPR-CAS9 was used to establish derivatives devoid of AXL (KO1 and K02; FIGS. 6G and 6H). Next, the animal study was repeated with the OX (FIG. 1C) and KO cells (FIG. 1D). As shown, cells overexpressing AXL evolved TKI resistance earlier than the parental cells. Conversely, KO cells displayed relatively slow tumorigenesis. Moreover, while control tumors relapsed, TKI-treated KO1 cells showed no relapses, although mice were followed for ˜3 months post implantation (FIG. 1D).

The observed absolute dependence of relapses on AXL proposed that adaptive activation of this RTK permits the emergence of TKI resistance. Consistently, analysis of a recently published patient dataset (21) confirmed that osimertinib treatment of EGFR tumors associated with AXL up-regulation (FIG. 1E; p<0.05). Assuming that AXL's role in resistance initiates early on, OX1, KO1 and parental cells were incubated with TKIs and 9 days later surviving Drug-Tolerant Persister (DTP) cells were quantified. As previously reported (8), very few cells survived this treatment. Nevertheless, AXL-overexpression increased this fraction (FIG. 1F) and fewer AXL-depleted cells survived treatment (FIG. 1G). In conclusion, AXL is activated or up-regulated in response to TKIs and supports survival of drug-treated cells; only very few AXL-ablated cells tolerate in-vitro treatments and no cells resist longer-term treatments in-vivo if AXL is not expressed.

### TKIs Increase DNA Breaks and Reduce Homologous Recombination Proficiency, but AXL Opposes these Effects

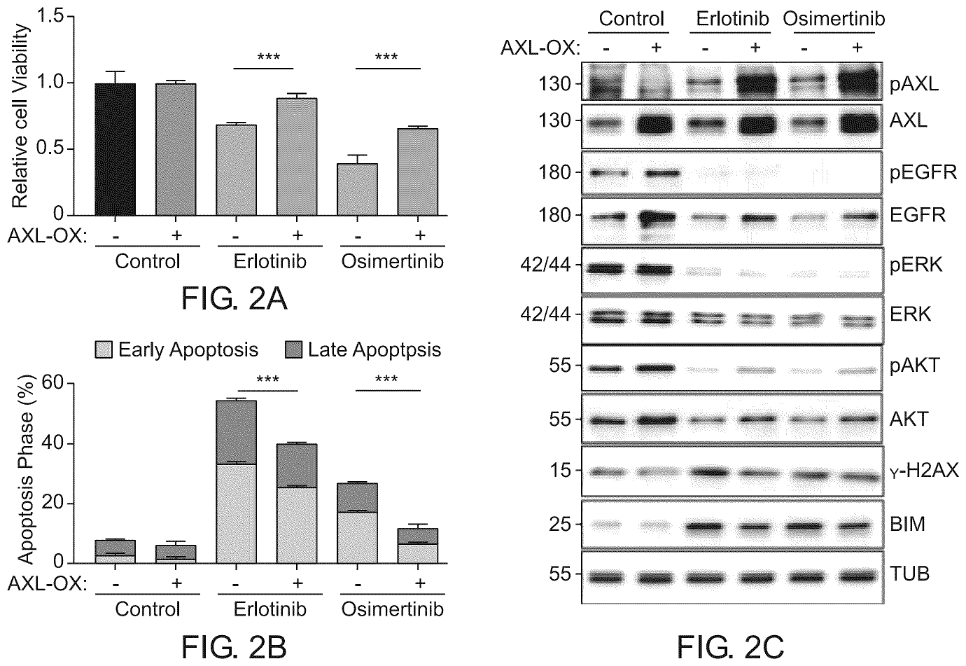

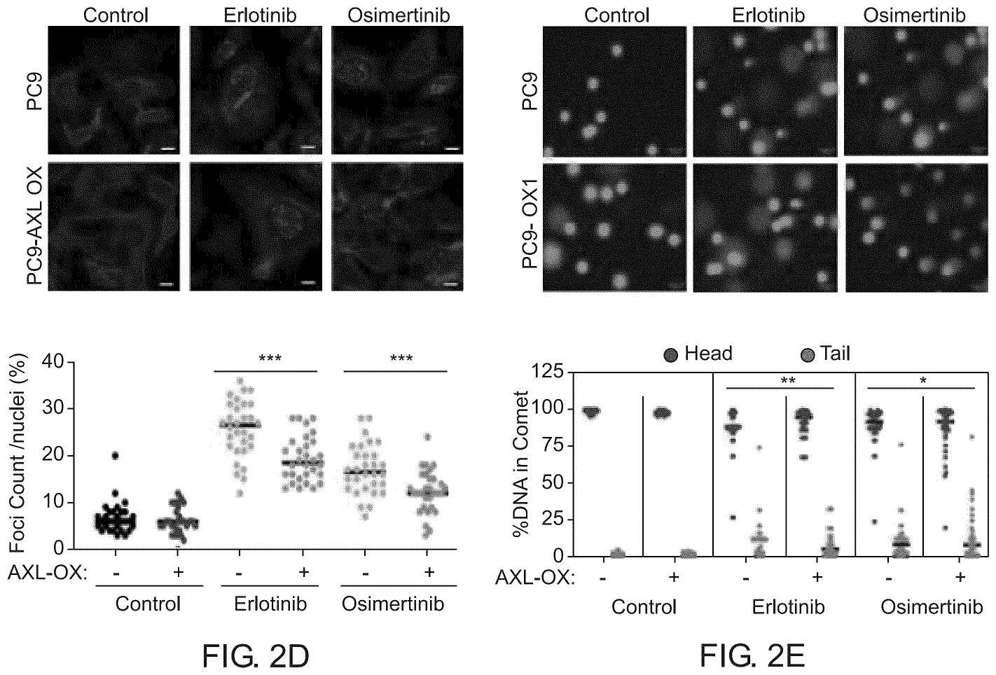

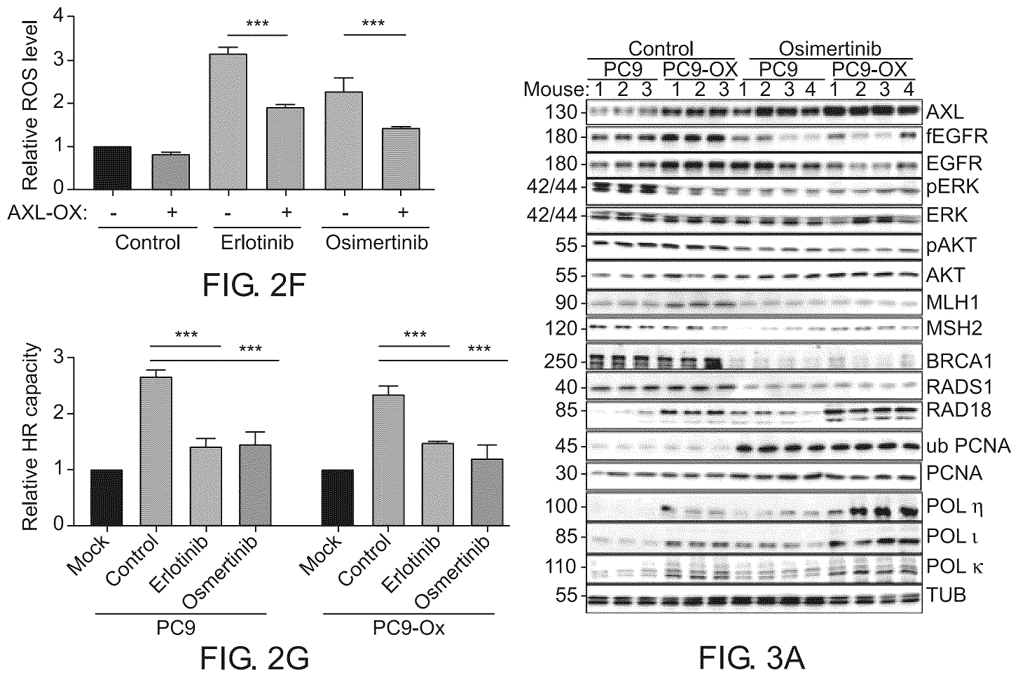

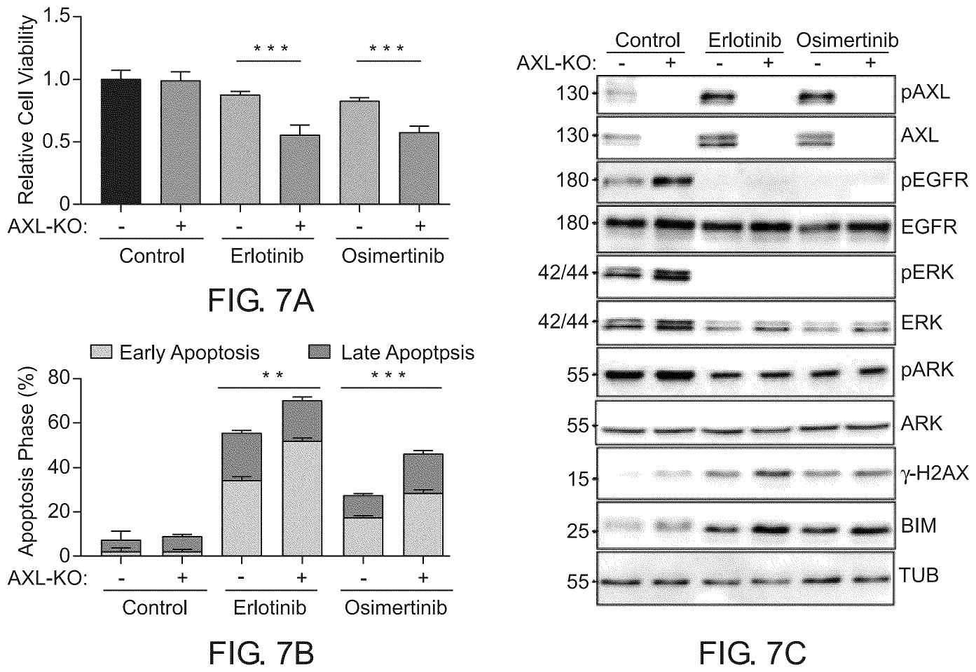

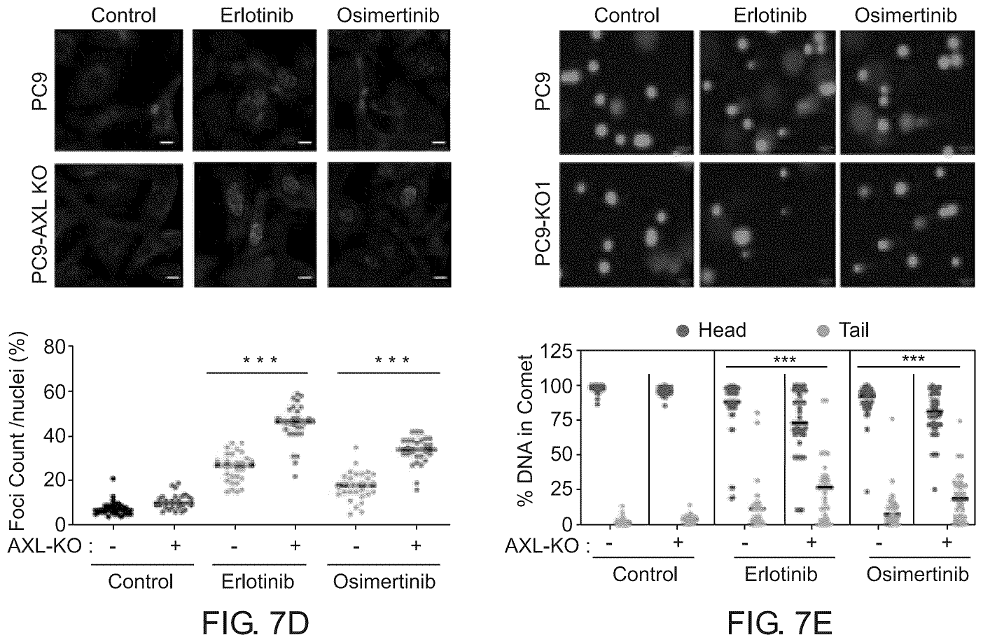

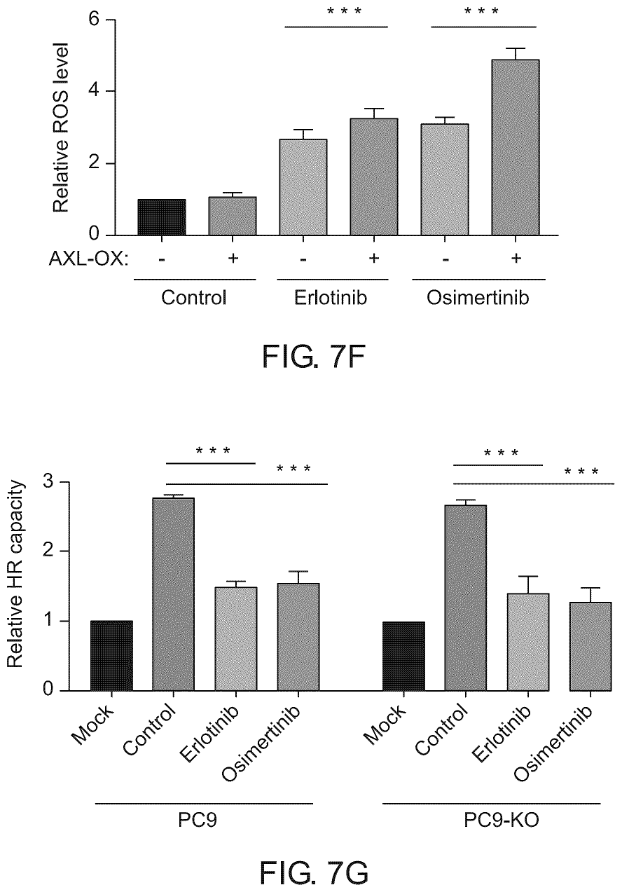

By inhibiting apoptosis, AXL confers resistance to multiple anti-cancer drugs (22). As expected, exposure of PC9 cells to TKIs increased apoptosis and reduced cell viability, but overexpression of AXL enhanced viability (FIG. 2A) and inhibited TKI-induced apoptosis (FIG. 2B). Analysis of KO cells (FIGS. 7A and 7B), confirmed the ability of AXL to inhibit apoptosis, likely due to AKT activation (FIGS. 2C and 7C). TKIs also increased gamma-H2A.X, which marks DNA double-strand breaks (DSB) (23), but AXL inhibited this effect (FIGS. 2C, 2D, 7C and 7D). By applying the alkaline comet assay, which monitors single- and double-strand DNA breaks (24), it was inferred that TKIs induce DNA fragmentation but AXL attenuates this effect (FIGS. 2E and 7E). In line with the ability of reactive oxygen species (ROS) to induce DSBs, TKI treatments increased ROS, but once again, AXL inhibited ROS production (FIGS. 2F and 7F). To ascertain that TKIs reduce DNA-repair competence, HR proficiency was evaluated by using a specific plasmid, pDRGFP/pCBASce-I (25), which confirmed marked reductions in HR proficiency post treatment with TKIs. However, this was not modulated by AXL (FIGS. 2G and 7G). In conclusion, TKI-induced inhibition of EGFR elevates ROS, increases DNA breaks and reduces HR proficiency, but AXL primarily inhibits these effects and reduces apoptosis.

### AXL Facilitates TKI-Induced Up-Regulation of RAD18 and Error-Prone DNA Polymerases

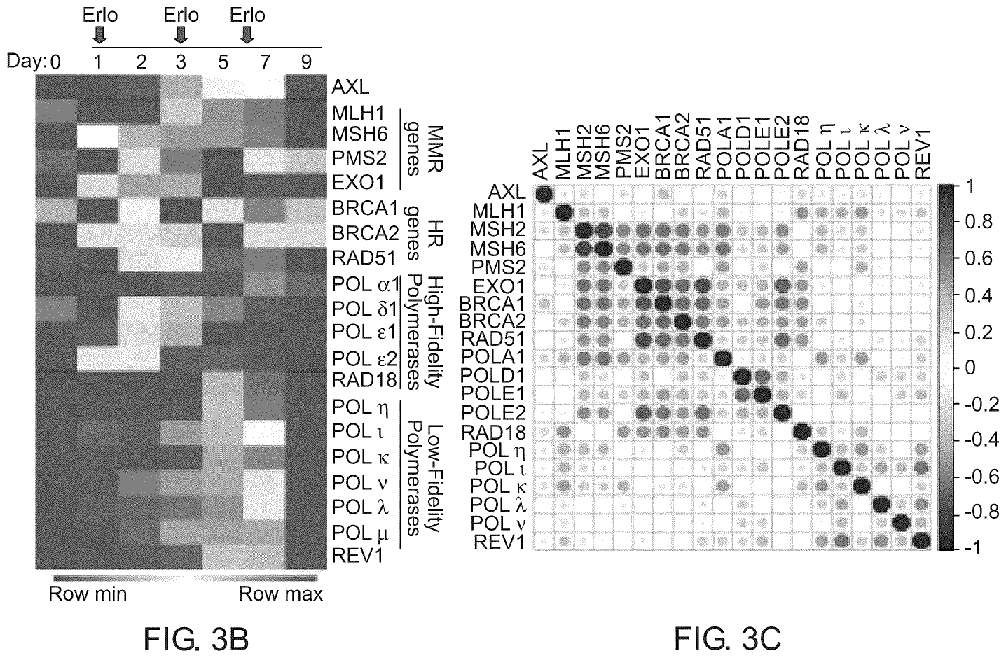

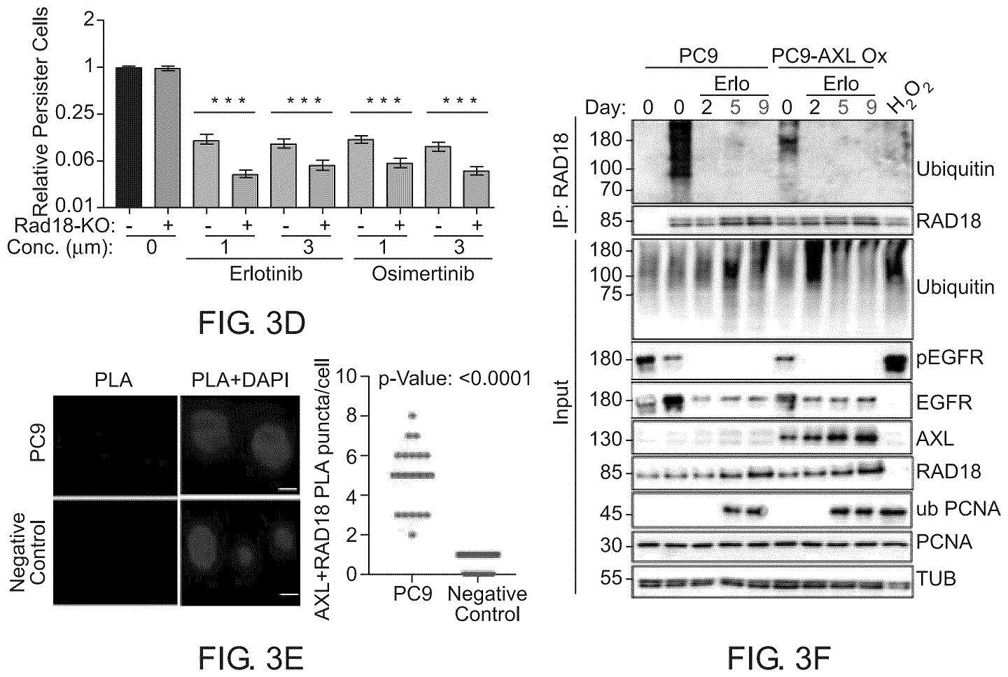

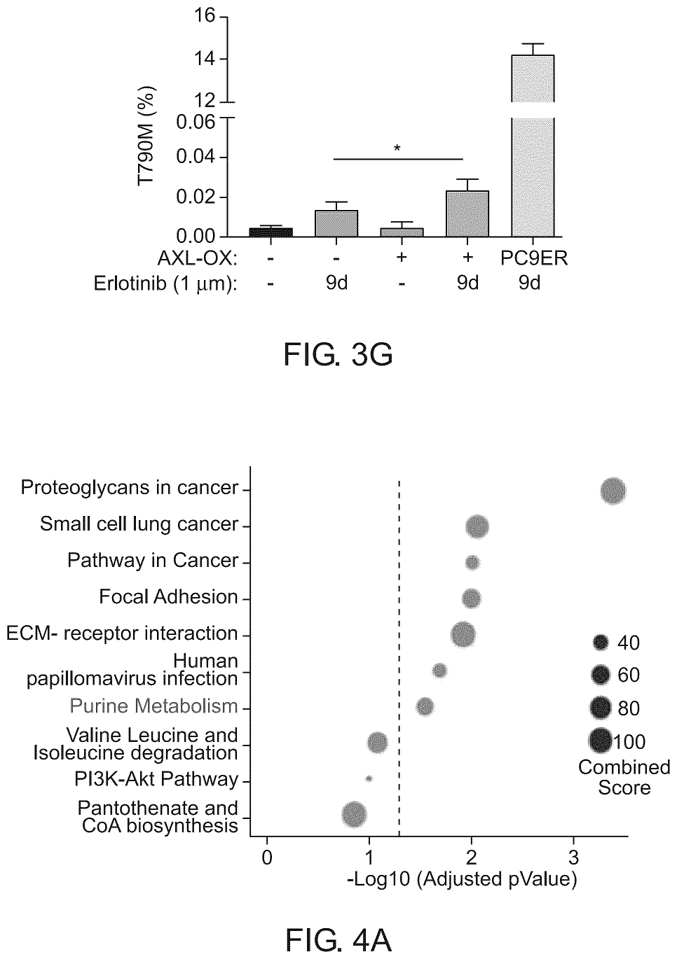

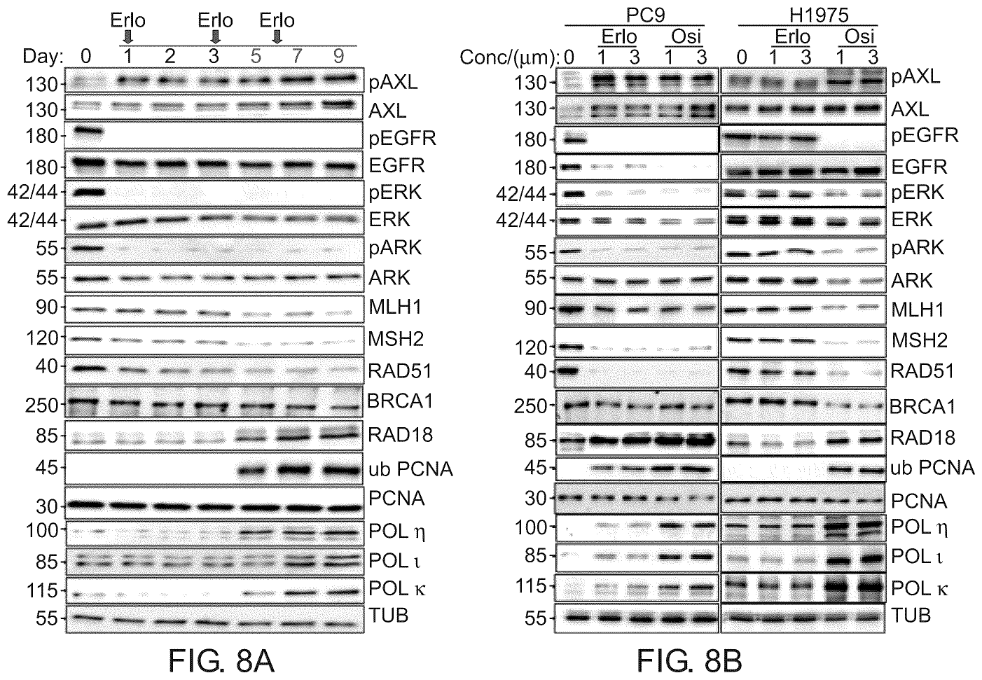

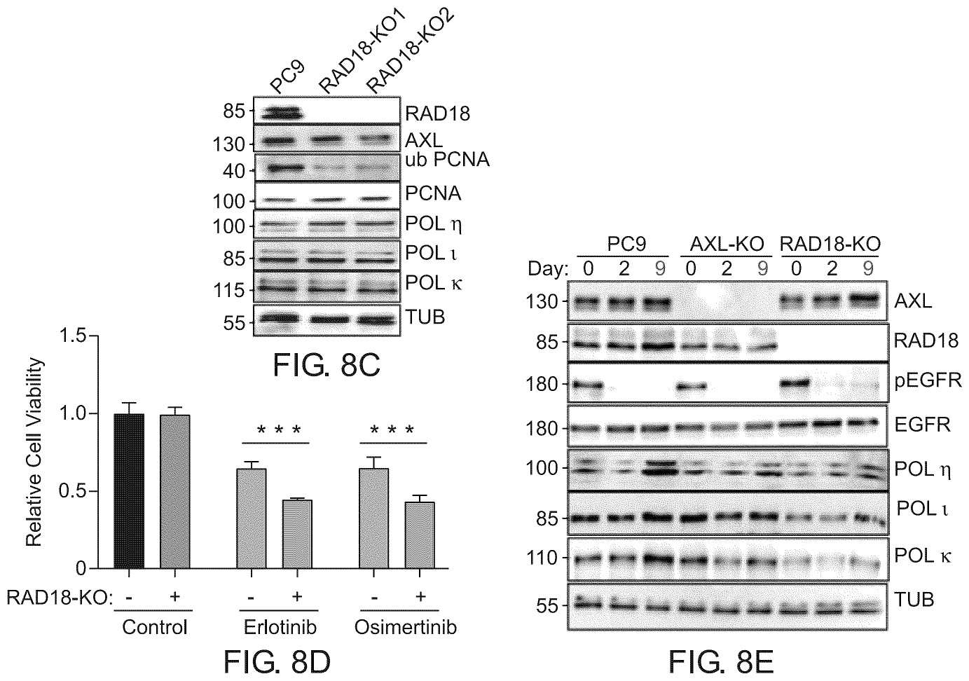

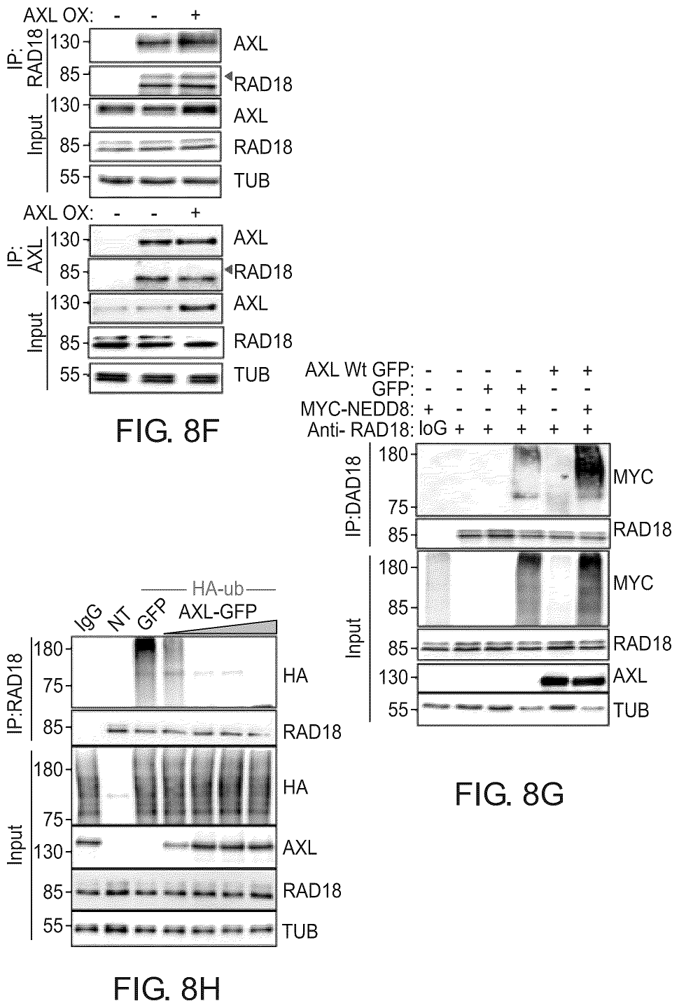

Because AXL inhibits rather than induces DNA breaks, potential involvement in base alterations was examined. Replication fidelity is influenced by DNA polymerases specialized in translesion synthesis (TLS): Following damage, the replicative polymerases are replaced by TLS polymerases (26), which collaborate with RAD18 (an E3 ubiquitin ligase) and enhance damage tolerance at the expense of replication fidelity (27). To examine AXL involvement, the present inventors extracted AXL-overexpressing and control tumors while they were relapsing under TKI treatment. In line with reduced HR proficiency, an AXL-independent reduction in RAD51 and BRCA1 was observed (FIG. 3A). In contrast, RAD18 and three error-prone polymerases (eta, iota and kappa) underwent up-regulation in TKI-treated tumors only if they overexpressed AXL. Interestingly, the ubiquitinated form of PCNA (proliferating cell nuclear antigen), a substrate of RAD18, was detected in all TKI-treated tumors. To unravel the origins of these alterations, qPCR was longitudinally applied (FIG. 3B) and immunoblotting (FIG. 8A) on DTPs. These assays detected late activation of AXL, which overlapped peaks of RAD18, ubiquitinated PCNA and seven low-fidelity polymerases. In parallel, four high-fidelity polymerases underwent downregulation, concurrent with inactivation of several HR and MMR genes. Treatment of H1975 cells (L858R and T790M EGFR), which are erlotinib-resistant and osimertinib-sensitive, indicated that resistance abolished RAD18 upregulation and other alterations (FIG. 8B). To quantify genomic alterations, untreated and TKI-treated tumors were compared by means of whole exome sequencing. This analysis detected a TKI-induced 2.31-(erlotinib) and 2.37 (osimertinib)-fold increase in base alterations in relapsing tumors and a larger increase, 2.42- or 2.52-fold, in the respective AXL-overexpressing tumors. To examine clinical relevance, transcriptomic data corresponding to 506 patients with lung adenocarcinoma was analyzed. Examining the correlation of AXL expression and that of 18 DNA-repair genes, a significant positive correlation between AXL and four Y-family polymerases (POLI, REV1, POLH and POLK; FIG. 3C) was found. In conclusion, AXL activation facilitates TKI-induced error-prone repair mechanisms and increase mutation rates in the PC9 tumor model.

### AXL Binds with and Activates RAD18 by Means of Neddylation

RAD18-mediated mono-ubiquitination of PCNA is critical for TLS (28); in response to damage, Ub₁-PCNA recruits Y-family polymerases to stalled replication forks (29). To address molecular interactions, PC9 derivatives lacking RAD18 were established (RAD18-KO; FIG. 8C). These cells displayed reduced viability when treated with TKIs (FIG. 8D) and gave rise to fewer DTPs (FIG. 3D), which exhibited compromised up-regulation of Y-family polymerases, similar to AXL-KO cells (FIG. 8E). Next, the present inventors tested whether AXL and RAD18 form complexes, a possibility previously raised on the basis of mass-spectrometry (30)). Both co-immunoprecipitation (FIG. 8F) and Proximity Ligation Assays (PLA; FIG. 3E) revealed physical interactions and detected the complexes in nuclei. Notably, the cleaved cytoplasmic domain of AXL can translocate into the nucleus via a nuclear localization sequence (31).

Because neddylation of RAD18 inhibits its ubiquitination and enhances interactions with PCNA (32), we predicted that AXL regulates this process. Overexpression of AXL enhanced neddylation (FIG. 8G) and reduced ubiquitination (FIG. 8H) of RAD18 in HEK293 cells. Correspondingly, when assayed in DTPs, both erlotinib treatment and AXL overexpression reduced ubiquitination of RAD18, in similarity to H202, and increased ubiquitination of PCNA (FIG. 3F), implying augmented TLS. Ultimately, these effects are expected to increase adaptive mutability. To test this, DTPs were subjected to digital PCR analysis of EGFR. This assay demonstrated that erlotinib enhanced emergence of T790M, the most prevalent resistance-causing mutation (33, 34), and AXL overexpression further enhanced this effect (FIG. 3G). In conclusion, AXL forms physical complexes with RAD18 and activates this E3 ligase by means of enhancing neddylation and inhibiting ubiquitination. The resulting active form of RAD18 initiates ubiquitination of PCNA, thereby promotes TLS and adaptive mutability.

### Along with Metastasis, AXL Enhances Purine Metabolism, which Might Propel Mutations

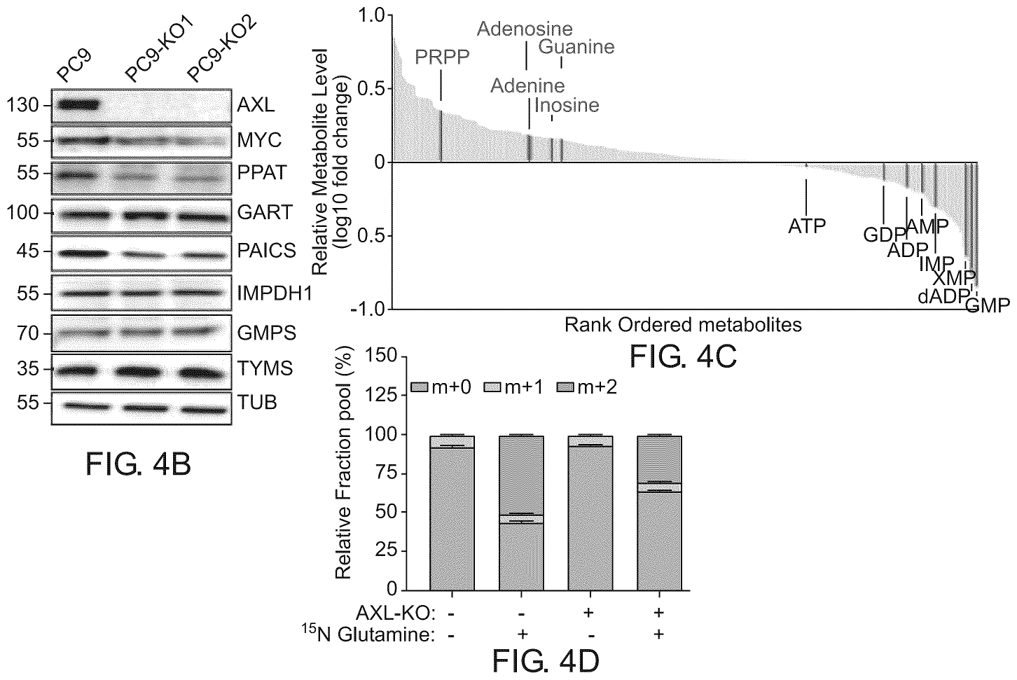

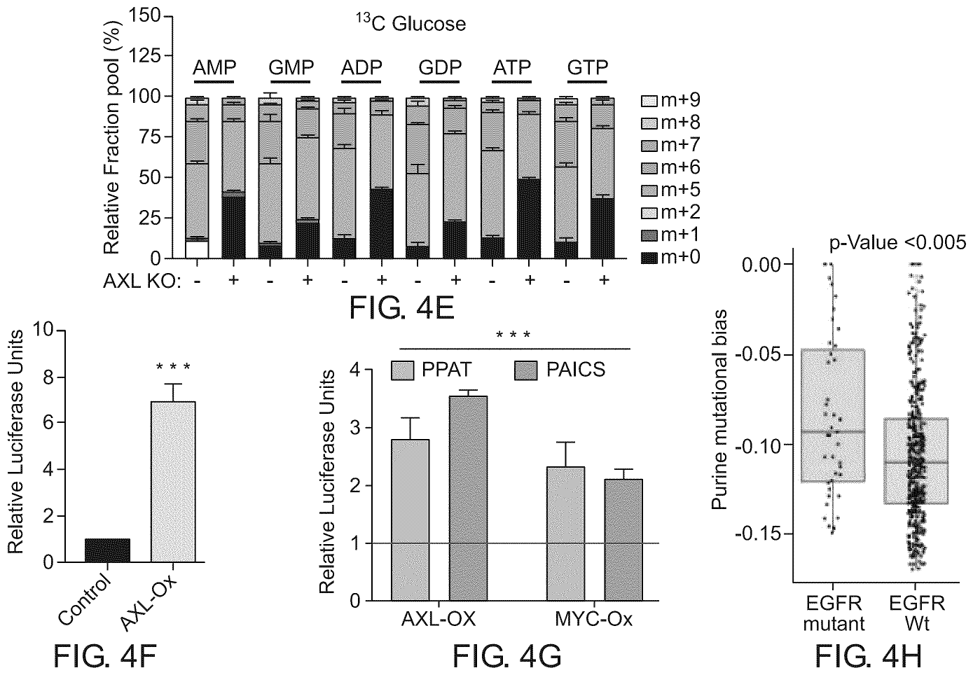

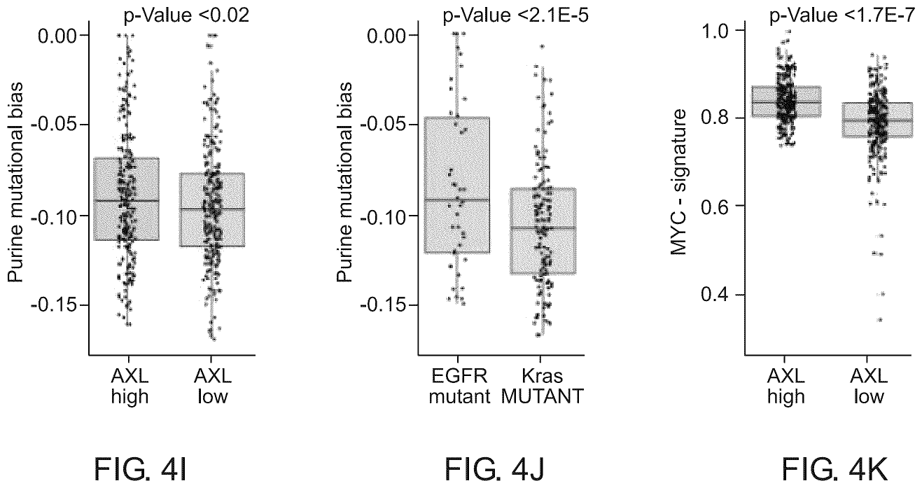

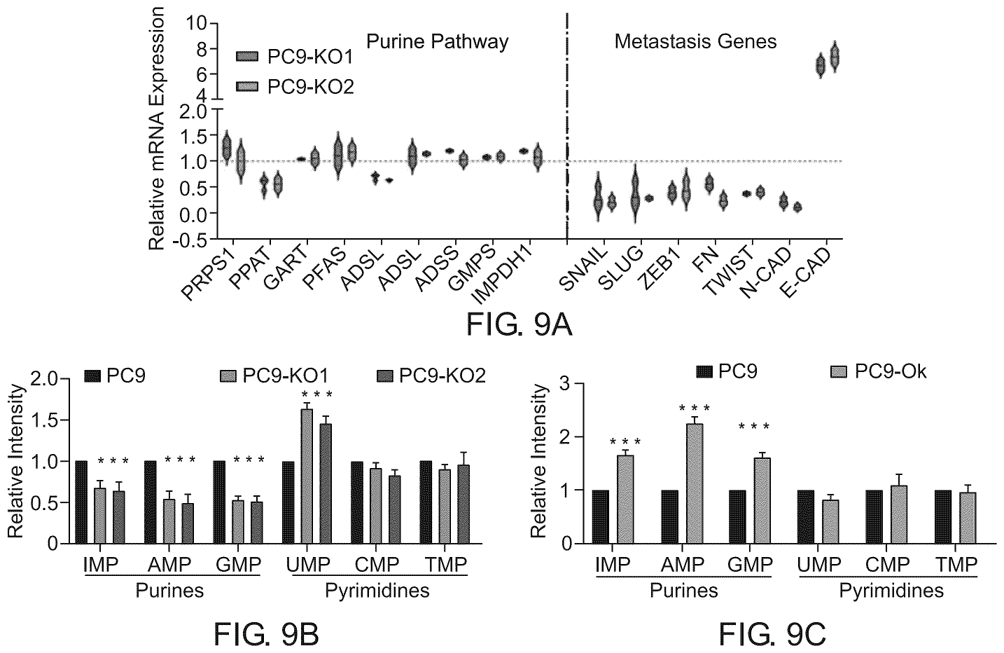

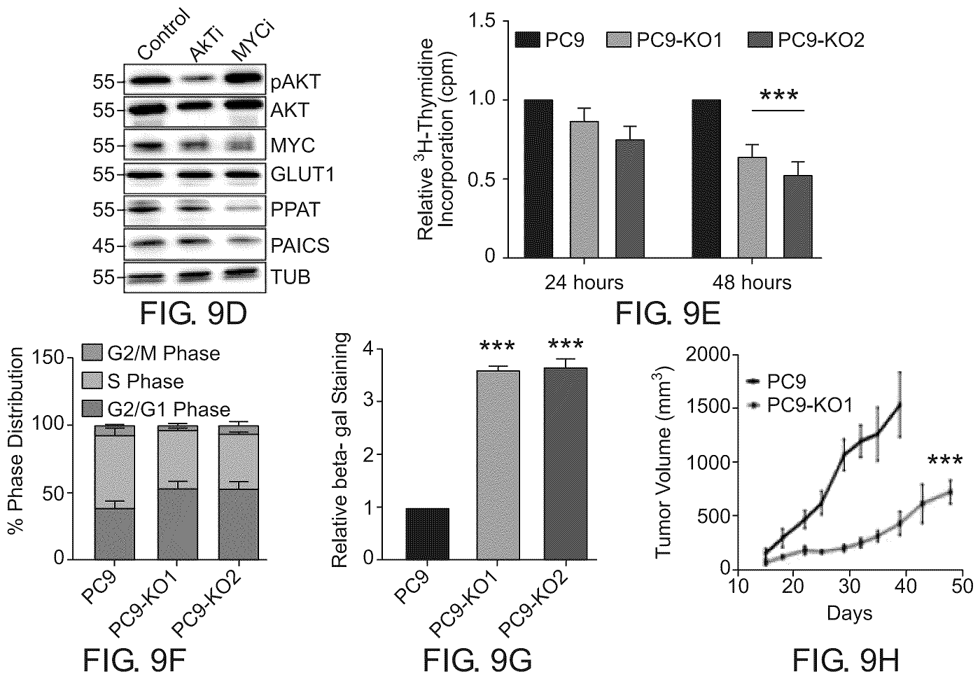

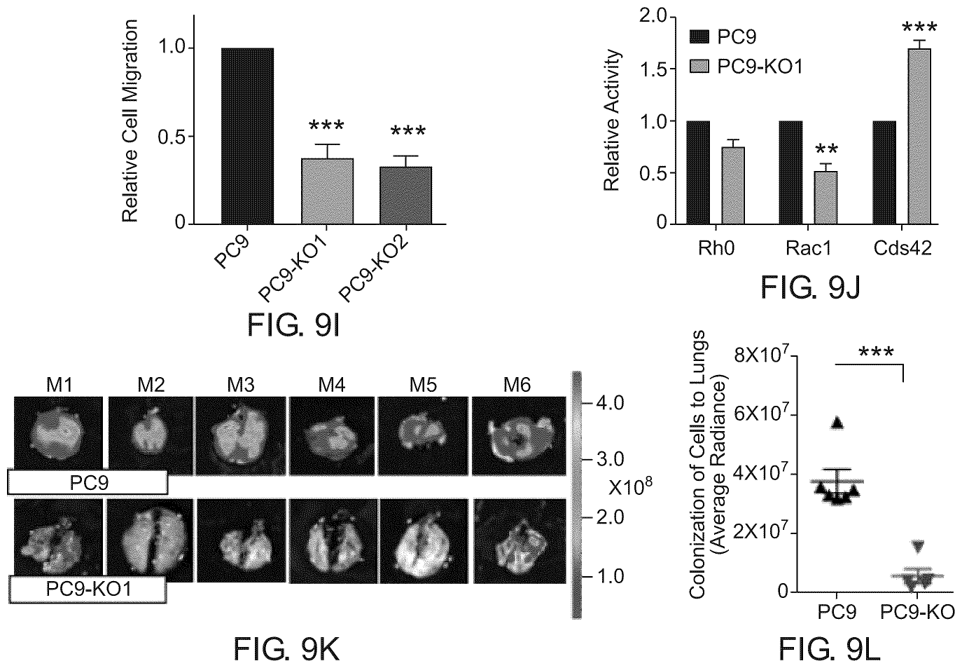

Next, RNA isolated from naïve and AXL-KO cells was sequenced and pathway enrichment analysis was performed (FIG. 4A). This uncovered primarily two groups of differentially regulated transcripts, one involved in metastasis and the other in nucleotide metabolism, mainly the inter-connected purine, histidine and glutamine pathways. PCR analysis confirmed the alterations in the expression of genes engaged in either EMT or purine metabolism (FIG. 9A), including phosphoribosyl pyrophosphate amidotransferase (PPAT) and PAICS (phosphoribosylaminoimidazole carboxylase and phosphoribosylaminoimidazolesuccinocarboxamide synthase), which was validated using immunoblots and PCR (FIGS. 4B and 9A). Notably, high-fidelity DNA replication depends on the cellular pools of deoxyribonucleotides (35). The pools influence DNA polymerase proofreading (36), as well as initiate mutator phenotypes due to unbalanced pyrimidine and purine bases (37, 38). To test these effects of AXL, extracts of KO and parental cells were subjected to global small molecule mass spectrometry (FIG. 4C). Among the top differential hits were metabolites in the de novo purine synthesis pathway, such as guanosine 5′-monophosphate (GMP) and inosine 5′-monophosphate (IMP), whose levels were decreased, along with increased levels of adenine, guanine and adenosine. Reduced levels of IMP, AMP and GMP were confirmed using targeted mass spectrometry (FIG. 9B), and conversely, these metabolites displayed increased levels in AXL-overexpressing cells (FIG. 9C).

To ascertain that de novo purine synthesis is controlled by AXL, targeted tandem mass spectrometry (LC-MS/MS) was used to measure the relative flux of stable isotope-labeled [amide-15N]glutamine, which is incorporated into the purine ring at two positions. Purine biosynthesis rates were evaluated by measuring isotope incorporation into IMP, as described (39). This analysis showed that the doubly labeled form of IMP, m+2, was dominant after incubating naïve PC9 cells with [amide-15N]glutamine (FIG. 4D). While m+1 did not significantly change in KO cells, these cells displayed reduced IMP m+2, in line with the ability of AXL to accelerate de novo purine synthesis. Incorporation of 13C arising on [11-13C]glucose, into purine nucleotides was also measured. In comparison to control cells, KO displayed lower m+2, m+6, m+7 and m+8 of the mono-, di- and tri-nucleotides, along with higher fractions of m+0 (FIG. 4E), further indicating that AXL can boost purine synthesis. Additional analyses attributed the underlying mechanism to the ability of AXL to activate MYC, a well-characterized transcriptional target of AXL (40): overexpression of AXL stimulated a MYC reporter (FIG. 4F), while a MYC inhibitor reduced PPAT and PAICS (FIG. 9D) and both AXL and MYC activated the corresponding reporters (FIG. 4G).

Taken together, these results testify that in response to TKIs, AXL levels are enhanced and transcriptionally activate MYC, which upregulates nucleotide metabolism and increases purine mutational bias (PuMB) (41). Consistent with this observation, it was found that PuMB scores in lung cancer tumors (TCGA; 506 patients) are associated with tumors harboring EGFR mutants versus wildtype EGFR tumors (FIG. 4H), and additionally, that high scores are associated with AXL-overexpressing tumors (FIG. 4I). These results are further testified by grouping lung cancers into tumors expressing a mutant form of EGFR or the larger group harboring RAS mutations, showing that the EGFR group has significantly higher PuMB levels (FIG. 4J). Consistent with involvement of the AXL-to-MYC axis, a core MYC gene expression signature (42) is strongly correlated with high levels of AXL in lung tumors (FIG. 4K). In conclusion, treatments making use of TKIs activate AXL and MYC, thereby unbalance nucleotide metabolism and increase purine-biased adaptive mutability.

Along with downregulated purine biosynthesis, AXL-KO cells exhibited altered expression of genes involved in cell migration and proliferation. In line with this, AXL-KO cells exhibited reduced rates of DNA synthesis, decreased S-phase fractions and increased senescence (FIGS. 9E-9G). In addition, these cells displayed relatively slow tumorigenic growth (FIG. 9H) and reduced rates of migration (FIG. 9I), as well as low levels of the active, GTP-loaded forms of RAC1 and CDC42 (FIG. 9J), which support migration. Consistently, luciferase-tagged AXL-KO cells severely lost the ability to colonize lungs following injection into mouse tail veins (FIGS. 9K and 9L). In summary, these results portray a dual function of AXL: a master regulator of purines and mutability, as well as a promoter of cell proliferation and metastasis.

### A New Anti-AXL Antibody Durably Inhibits Resistance to Osimertinib

Since AXL has emerged as a facilitator of TKI-induced mutator phenotypes, anti-AXL mAbs were generated. One antibody, mAb654, was selected because it inhibited pAKT and DNA synthesis (FIGS. 10A and 10B). Because combining cetuximab, an anti-EGFR mAb, and TKIs delayed relapses of drug-resistant models (43), the present inventors tested various combinations of cetuximab, osimertinib and mAb654. Unlike the ˜60% viability reduction observed with PC9 and H1975 cells treated with TKIs, both cetuximab and mAb654 only weakly inhibited viability, but the triple combination achieved 80% inhibition (FIGS. 10C and 10D). This cooperative effect was attributable to the ability of the triplet to enhance apoptosis (FIG. 10E), as well as boost ROS production (FIG. 10F). Moreover, the triplet downregulated AXL, pAKT, PPAT, RAD18 and several low-fidelity polymerases (FIG. 10G). Ultimately, these attributes translated to strong diminution of DTPs (FIGS. 10H and 10I).

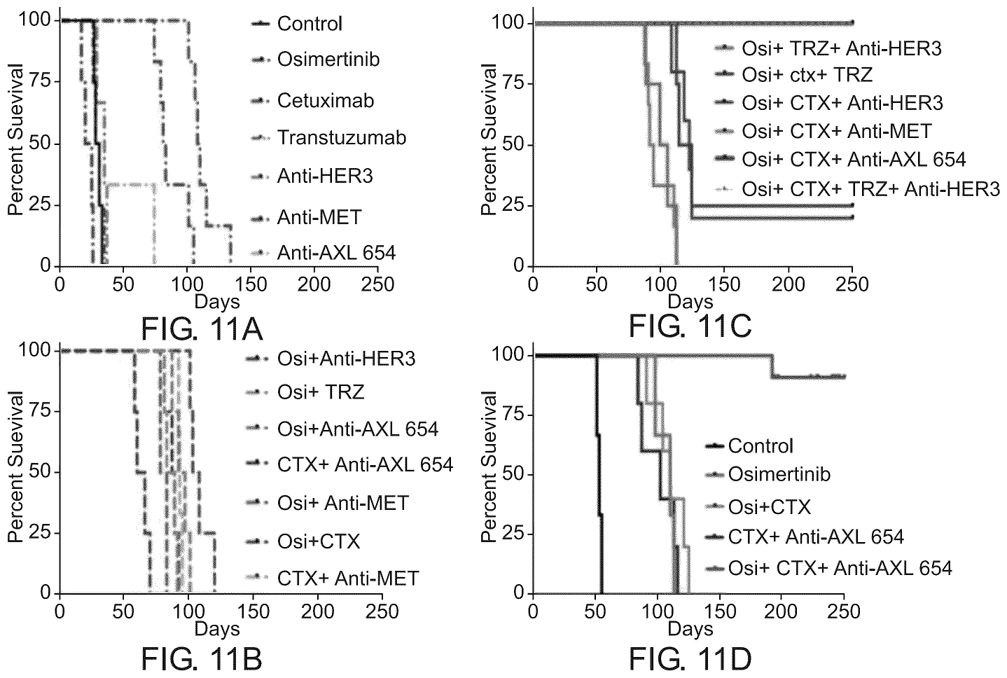

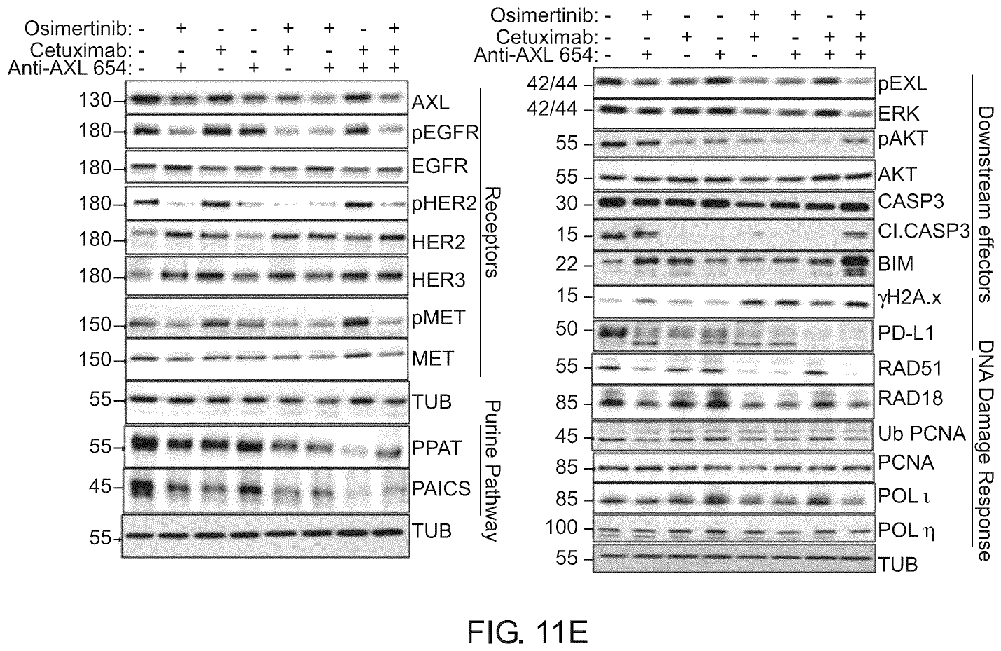

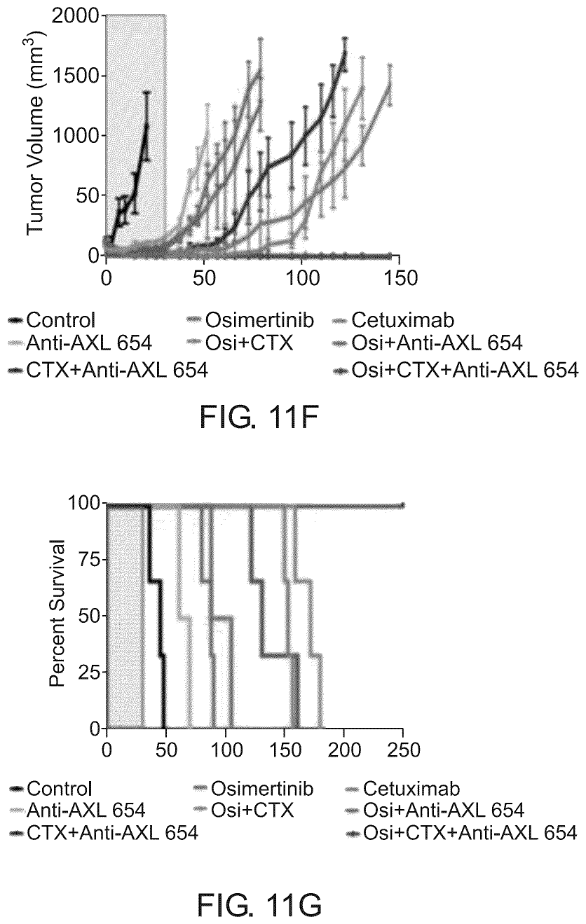

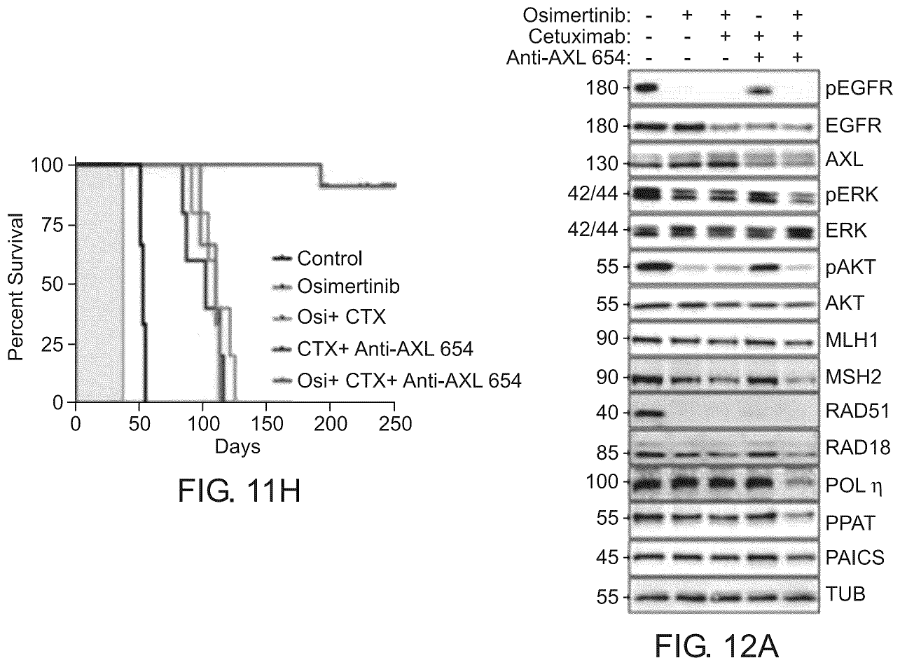

The triplet's ability to nearly eliminate DTPs predicted inhibition of tumor relapses. PC9 cells were implanted in athymic mice and, when tumors became palpable, mice were randomized into groups (n=8) that were treated with mAbs specific to AXL, EGFR, HER2, HER3, or MET (FIG. 5A). All treatments were discontinued on day 30, but animals continued to be monitored. Of the mAbs used, cetuximab better delayed relapses (FIG. 5B; see survival curves in FIGS. 11A-11C). Next, drug doublets were tested and it was found that cetuximab+osimertinib achieved longest delays (FIG. 5B). Remarkably, although mAb654+osimertinib was inferior, the anti-AXL mAb achieved superior effects when combined with both cetuximab and osimertinib (FIG. 5C). Importantly, similar results were observed when treating H1975 tumors (FIG. 11F and 11G). In fact, despite short treatments with the triplet (30 days), no relapses were observed up to 4 months later. Taken together, these observations proposed that transient inhibition of the AXL-dependent genetic switch that transforms persisters to resisters can cure the PC9 animal model.

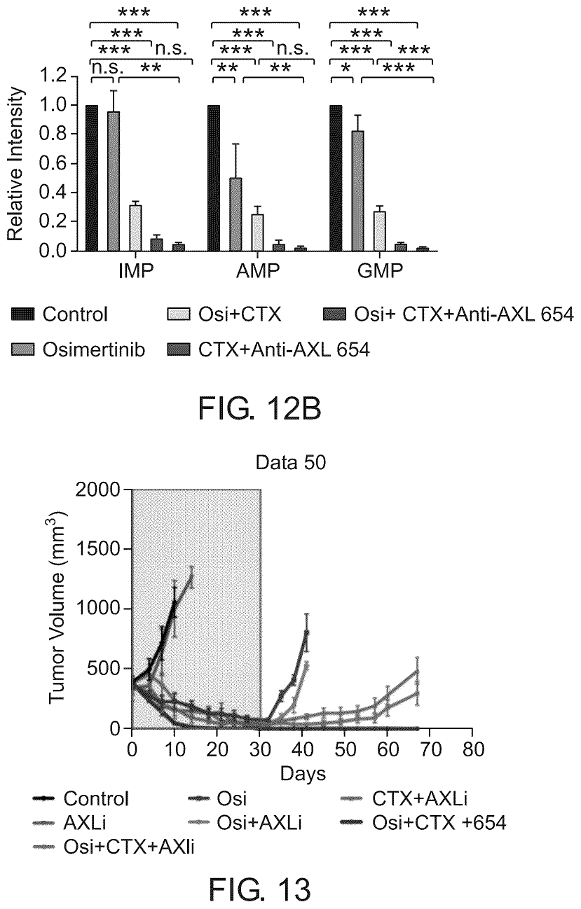

Two approaches were undertaken to verify the durable effect of the relatively short AXL blockade: (i) analysis of patient-derived xenografts (PDX), which better represent tumor heterogeneity (44), and (ii) testing impact on the clinically more relevant, pre-established state of resistance. Using TM00193, a PDX expressing EGFR E746-A750 deletion, the ability of mAb654, in combination with anti-EGFR drugs, to overcome resistance (FIG. 5D and FIG. 11H). The other approach simulated evolvement of clinical resistance: pre-established treatment-naïve PC9 tumors were firstly treated with osimertinib. Under these conditions, tumors initially regress but later relapse. Once tumors regained the initial volume, animals were randomized into groups that were treated with combinations of cetuximab, osimertinib and mAb654. All groups, save the one treated with the triplet, experienced relapses (FIG. 5E). In contrast, the triplet-treated group achieved full regressions and remained tumor free following cessation of all treatments (FIGS. 5E1-5E6; each panel represents one animal). Tumor extracts obtained early after treatment onset confirmed that the triplet enhanced apoptosis and reduced proteins involved in TLS and purine metabolism (FIG. 11E and 12A), and as expected diminished IMP, AMP and GMP (FIG. 12B). Taken together, these results established the ability of short treatments with the anti-AXL antibody to durably overcome both de novo and pre-established resistance in several animal models.

In summary, the adaptive response of lung tumors to EGFR TKIs entails up-regulation of several RTKs, including AXL. Preventing this response by ablating AXL reduced DTP numbers and completely prevented tumor relapses. It was found that TKI-induced up-regulation of AXL is associated with two mutator phenotypes: (i) up-regulation of RAD18 and several TLS polymerases, along with downregulation of replicative DNA polymerases, and (ii) activation of MYC, which imbalances intracellular nucleotide pools. Mechanistically, both AXL and TKIs activate RAD18 by inducing neddylation and preventing RAD18 ubiquitination, thereby harmonizing the polymerase switch. Analyses of patient data confirmed that AXL expression associates with both increased purine mutational bias and high abundance of mutation-prone polymerases. In line with AXL-dependent conversion of drug-tolerant persisters to resisters, a new anti-AXL antibody we generated completely inhibited resistance to TKIs when transiently delivered to tumor-bearing animals.

### A Combination of an AXL-Specific Kinase Inhibitor, Bemcentinib, and an Anti-EGFR Antibody Delays Relapses of a Lung Cancer Model that Acquires Resistance to Osimertinib

PC9 cells (3×10⁶) were implanted in CD1-nu/nu mice. When tumors became palpable, mice were randomized into groups of 7 animals each. Each group was treated (hatched area, 30 days) with the indicated antibodies (total dose: 0.2 mg/mouse/injection) once every three days, or daily with osimertinib (5 mg/kg/day) or the AXL-specific kinase inhibitor (bemcentinib; 75 mg/kg/day). As illustrated in FIG. 13, the combination of cetuximab and bemcentinib was effective at reducing tumor volume relapses in a cancer model that acquires resistance to osimertinib.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### References

1. M. Radman, SOS repair hypothesis: phenomenology of an inducible DNA repair which is accompanied by mutagenesis. Basic Life Sci 5A, 355-367 (1975).
2. Z. Baharoglu, D. Mazel, SOS, the formidable strategy of bacteria against aggressions. FEMS Microbiol Rev 38, 1126-1145 (2014).
3. D. M. Fitzgerald, P. J. Hastings, S. M. Rosenberg, Stress-Induced Mutagenesis: Implications in Cancer and Drug Resistance. Annu Rev Cancer Biol 1, 119-140 (2017).
4. M. Russo et al., Adaptive mutability of colorectal cancers in response to targeted therapies. Science 366, 1473-1480 (2019).
5. L. A. Garraway, P. A. Janne, Circumventing cancer drug resistance in the era of personalized medicine. Cancer Discov 2, 214-226 (2012).
6. D. J. Konieczkowski, C. M. Johannessen, L. A. Garraway, A Convergence-Based Framework for Cancer Drug Resistance. Cancer Cell 33, 801-815 (2018).
7. N. Q. Balaban, J. Merrin, R. Chait, L. Kowalik, S. Leibler, Bacterial persistence as a phenotypic switch. Science 305, 1622-1625 (2004).
8. S. V. Sharma et al., A chromatin-mediated reversible drug-tolerant state in cancer cell subpopulations. Cell 141, 69-80 (2010).
9. W. Pao et al., Acquired resistance of lung adenocarcinomas to gefitinib or erlotinib is associated with a second mutation in the EGFR kinase domain. PLoS Med 2, e73 (2005).
10. S. Kobayashi et al., EGFR mutation and resistance of non-small-cell lung cancer to gefitinib. N Engl J Med 352, 786-792 (2005).
11. G. R. Oxnard et al., New strategies in overcoming acquired resistance to epidermal growth factor receptor tyrosine kinase inhibitors in lung cancer. Clin Cancer Res 17, 5530-5537 (2011).
12. J. A. Engelman et al., MET amplification leads to gefitinib resistance in lung cancer by activating ERBB3 signaling. Science 316, 1039-1043 (2007).
13. K. R. Jakobsen, C. Demuth, B. S. Sorensen, A. L. Nielsen, The role of epithelial to mesenchymal transition in resistance to epidermal growth factor receptor tyrosine kinase inhibitors in non-small cell lung cancer. Translational lung cancer research 5, 172-182 (2016).
14. M. L. Sos et al., PTEN loss contributes to erlotinib resistance in EGFR-mutant lung cancer by activation of Akt and EGFR. Cancer Res 69, 3256-3261 (2009).
15. M. Mancini et al., An oligoclonal antibody durably overcomes resistance of lung cancer to third-generation EGFR inhibitors. EMBO Mol Med 10, 294-308 (2018).
16. D. Romaniello et al., A Combination of Approved Antibodies Overcomes Resistance of Lung Cancer to Osimertinib by Blocking Bypass Pathways. Clin Cancer Res 24, 5610-5621 (2018).
17. H. Taniguchi et al., AXL confers intrinsic resistance to osimertinib and advances the emergence of tolerant cells. Nat Commun 10, 259 (2019).
18. Z. Zhang et al., Activation of the AXL kinase causes resistance to EGFR-targeted therapy in lung cancer. Nature genetics 44, 852-860 (2012).
19. D. K. Graham, D. DeRyckere, K. D. Davies, H. S. Earp, The TAM family phosphatidylserine sensing receptor tyrosine kinases gone awry in cancer. Nat Rev Cancer 14, 769-785 (2014).
20. E. A. Carrera Silva et al., T cell-derived protein S engages TAM receptor signaling in dendritic cells to control the magnitude of the immune response. Immunity 39, 160-170 (2013).
21. N. Roper et al., Clonal Evolution and Heterogeneity of Osimertinib Acquired Resistance Mechanisms in EGFR Mutant Lung Cancer. Cell Rep Med 1, (2020).
22. M. Schoumacher, M. Burbridge, Key Roles of AXL and MER Receptor Tyrosine Kinases in Resistance to Multiple Anticancer Therapies. Curr Oncol Rep 19, 19 (2017).
23. J. Ji et al., Phosphorylated fraction of H2AX as a measurement for DNA damage in cancer cells and potential applications of a novel assay. PLoS One 12, e0171582 (2017).
24. P. Majtnerova, T. Rousar, An overview of apoptosis assays detecting DNA fragmentation. Mol Biol Rep 45, 1469-1478 (2018).
25. A. J. Pierce, R. D. Johnson, L. H. Thompson, M. Jasin, XRCC3 promotes homology-directed repair of DNA damage in mammalian cells. Genes Dev 13, 2633-2638 (1999).
26. W. Yang, Y. Gao, Translesion and Repair DNA Polymerases: Diverse Structure and Mechanism. Annu Rev Biochem 87, 239-261 (2018).
27. Y. Yang et al., Diverse roles of RAD18 and Y-family DNA polymerases in tumorigenesis. Cell Cycle 17, 833-843 (2018).
28. P. Stelter, H. D. Ulrich, Control of spontaneous and damage-induced mutagenesis by SUMO and ubiquitin conjugation. Nature 425, 188-191 (2003).
29. M. Bienko et al., Ubiquitin-binding domains in Y-family polymerases regulate translesion synthesis. Science 310, 1821-1824 (2005).
30. Y. Gao et al., A neomorphic cancer cell-specific role of MAGE-A4 in trans-lesion synthesis. Nat Commun 7, 12105 (2016).
31. Y. Lu et al., Regulated intramembrane proteolysis of the AXL receptor kinase generates an intracellular domain that localizes in the nucleus of cancer cells. Faseb J 31, 1382-1397 (2017).
32. J. Guan, X. Zheng, NEDDylation regulates RAD18 ubiquitination and localization in response to oxidative DNA damage. Bichem. Biophys. Res. Commun. 508, 1240-1244 (2018).
33. A. N. Hata et al., Tumor cells can follow distinct evolutionary paths to become resistant to epidermal growth factor receptor inhibition. Nature medicine 22, 262-269 (2016).
34. D. W. Bell et al., Inherited susceptibility to lung cancer may be associated with the T790M drug resistance mutation in EGFR. Nat Genet 37, 1315-1316 (2005).
35. T. T. Schmidt et al., Alterations in cellular metabolism triggered by URA7 or GLN3 inactivation cause imbalanced dNTP pools and increased mutagenesis. Proc Natl Acad Sci USA 114, E4442-E4451 (2017).
36. M. Huang, S. J. Elledge, Identification of RNR4, encoding a second essential small subunit of ribonucleotide reductase in Saccharomyces cerevisiae. Mol Cell Biol 17, 6105-6113 (1997).
37. A. C. Bester et al., Nucleotide deficiency promotes genomic instability in early stages of cancer development. Cell 145, 435-446 (2011).
38. J. S. Lee et al., Urea Cycle Dysregulation Generates Clinically Relevant Genomic and Biochemical Signatures. Cell 174, 1559-1570 e1522 (2018).
39. I. Ben-Sahra, G. Hoxhaj, S. J. H. Ricoult, J. M. Asara, B. D. Manning, mTORC1 induces purine synthesis through control of the mitochondrial tetrahydrofolate cycle. Science 351, 728-733 (2016).
40. J. Hong, S. Maacha, A. Belkhiri, Transcriptional upregulation of c-MYC by AXL confers epirubicin resistance in esophageal adenocarcinoma. Mol Oncol 12, 2191-2208 (2018).
41. R. Keshet et al., Targeting purine synthesis in ASS1-expressing tumors enhances the response to immune checkpoint inhibitors. Nature Cancer 1, 894-908 (2020).
42. S. Chandriani et al., A core MYC gene expression signature is prominent in basal-like breast cancer but only partially overlaps the core serum response. PLoS One 4, e6693 (2009).
43. V. Pirazzoli et al., Afatinib plus Cetuximab Delays Resistance Compared to Single-Agent Erlotinib or Afatinib in Mouse Models of TKI-Naive EGFR L858R-Induced Lung Adenocarcinoma. Clin Cancer Res 22, 426-435 (2016).
44. D. Siolas, G. J. Hannon, Patient-derived tumor xenografts: transforming clinical samples into mouse models. Cancer Res 73, 5315-5319 (2013).
45. Z. E. Stine, Z. E. Walton, B. J. Altman, A. L. Hsieh, C. V. Dang, MYC, Metabolism, and Cancer. Cancer Discov 5, 1024-1039 (2015).
46. C. K. Mathews, DNA precursor metabolism and genomic stability. FASEB J 20, 1300-1314 (2006).
47. T. Nishimura et al., Cancer stem-like properties and gefitinib resistance are dependent on purine synthetic metabolism mediated by the mitochondrial enzyme MTHFD2. Oncogene 38, 2464-2481 (2019).
48. L. Magnani et al., Genome-wide reprogramming of the chromatin landscape underlies endocrine therapy resistance in breast cancer. Proc Natl Acad Sci U S A110, E1490-1499 (2013).
49. J. Muller et al., Low MITF/AXL ratio predicts early resistance to multiple targeted drugs in melanoma. Nat Commun 5, 5712 (2014).
50. M. Badarni et al., Repression of AXL expression by AP-1/JNK blockage overcomes resistance to PI3Ka therapy. JCI Insight 5, (2019).
51. R. Wang et al., Transient IGF-1R inhibition combined with osimertinib eradicates AXL-low expressing EGFR mutated lung cancer. Nat Commun 11, 4607 (2020).
52. X. Chen et al., An immunological approach reveals biological differences between the two NDF/heregulin receptors, ErbB-3 and ErbB-4. J Biol Chem 271, 7620-7629 (1996).
53. S. Malitsky et al., Viral infection of the marine alga Emiliania huxleyi triggers lipidome remodeling and induces the production of highly saturated triacylglycerol. New Phytol 210, 88-96 (2016).
54. D. A. Jaitin et al., Massively parallel single-cell RNA-seq for marker-free decomposition of tissues into cell types. Science 343, 776-779 (2014).
55. R. Kohen et al., UTAP: User-friendly Transcriptome Analysis Pipeline. BMC Bioinformatics 20, 154 (2019).
56. M. I. Love, W. Huber, S. Anders, Moderated estimation of fold change and dispersion for RNA-seq data with DESeq2. Genome Biol 15, 550 (2014).
57. E. Y. Chen et al., Enrichr: interactive and collaborative HTML5 gene list enrichment analysis tool. BMC Bioinformatics 14, 128 (2013).
58. M. V. Kuleshov et al., Enrichr: a comprehensive gene set enrichment analysis web server 2016 update. Nucleic Acids Res 44, W90-97 (2016).
59. R. Huang et al., The NCATS BioPlanet—An Integrated Platform for Exploring the Universe of Cellular Signaling Pathways for Toxicology, Systems Biology, and Chemical Genomics. Front Pharmacol 10, 445 (2019).

What is claimed is:

1. A method of treating cancer in a subject in need thereof comprising administering to the subject a therapeutically effective amount of:
(i) an anti-epidermal growth factor receptor (anti-EGFR) antibody;
(ii) a small molecule tyrosine kinase inhibitor (TKI); and
(iii) an AXL inhibitor,
wherein cancer cells of said subject express an EGFR having a mutation in a kinase domain of said receptor, thereby treating the cancer in the subject.

2. The method of claim 1, wherein said mutation comprises a substitution of Threonine to Methionine at position 790 (T790M).

3. The method of claim 1, wherein said cancer is resistant to said TKI when provided as a monotherapy.

4. The method of claim 3, wherein said treating is a first line treatment.

5. The method of claim 3, wherein said treating is a second line treatment.

6. The method of claim 1, wherein said TKI is provided below gold standard dosing as a single agent.

7. The method of claim 1, wherein said cancer is lung cancer.

8. The method of claim 7, wherein said lung cancer is non-small cell lung cancer (NSCLC).

9. The method of claim 1, wherein said anti-EGFR antibody is cetuximab.

10. The method of claim 1, wherein said TKI is selected from the group consisting of osimertinib, perlitinib (EKB-569), neratinib (HKI-272), canertinib (CI-1033), vandetanib (ZD6474), afatinib, dacomitinib, rociletinib (CO-1686), HM61713, Gefitinib, Erlotinib, Icotinib and WZ4002.

11. The method of claim 1, wherein said AXL inhibitor is an antibody which specifically binds to AXL or a small molecule inhibitor.

12. The method of claim 1, wherein said AXL inhibitor is an antibody which comprises an antigen recognition domain having complementarity determining region (CDR) amino acid sequences as set forth in: SEQ ID NOs: 1 (CDR1), SAF (CDR2) and 3 (CDR3), sequentially arranged from N to C on a light chain of the antibody and SEQ ID NOs: 4 (CDR1), 5 (CDR2) and 6 (CDR3), sequentially arranged from N to C on a heavy chain of the antibody.

13. The method of claim 1, further comprising confirming that the cancer cells of the subject express said mutation prior to said administering.

14. A method of treating lung cancer in a subject in need thereof comprising administering to the subject a therapeutically effective amount of:
(i) an anti-epidermal growth factor receptor (anti-EGFR) antibody; and
(ii) an AXL inhibitor,
wherein cancer cells of said subject express an EGFR having a mutation in a kinase domain of said receptor, wherein said cancer is resistant to a TKI when provided as a monotherapy, thereby treating the lung cancer in the subject.

15. The method of claim 14, wherein said anti-EGFR antibody is cetuximab.

16. The method of claim 14, wherein said AXL inhibitor is an antibody which specifically binds to AXL.

17. The method of claim 14, wherein said AXL inhibitor is a small molecule inhibitor.

18. The method of claim 14, wherein said lung cancer is non-small cell lung cancer (NSCLC).

19. A pharmaceutical composition comprising an antibody that specifically binds to AXL as an active agent and a pharmaceutically acceptable carrier, said antibody comprising an antigen recognition domain having complementarity determining region (CDR) amino acid sequences as set forth in: SEQ ID NOs: 1 (CDR1), SAF (CDR2) and 3 (CDR3), sequentially arranged from N to C on a light chain of the antibody) and SEQ ID NOs: 4 (CDR1), 5 (CDR2) and 6 (CDR3), sequentially arranged from N to C on a heavy chain of the antibody.

20. The pharmaceutical composition of claim 19, further comprising an anti-epidermal growth factor receptor (anti-EGFR) antibody and/or a tyrosine kinase inhibitor (TKI).
